(19) United States
(12) Patent Application Publication
(10) Pub. No.: US 2011/0229427 A1
(43) Pub. Date: Sep. 22, 2011

(54) Phosphoric Acid Esters Containing Phosphorus Atoms Bridged By Polyol Units

(75) Inventors: Peter Klug, Grossostheim (DE); Franz-Xaver Scherl, Burgkirchen (DE); Waltraud Simsch, Kelkheim (DE); Adelgunde Oberhauser, Neuoetting (DE)

(73) Assignee: CLARIANT FINANCE (BVI) LIMITED, Tortola (VG)

(21) Appl. No.: 12/671,792

(22) PCT Filed: Jul. 29, 2008
(86) PCT No.: PCT/EP2008/006220

§ 371 Date: Jul. 29, 2010

(30) Foreign Application Priority Data
Aug. 2, 2007 (DE) 10 2007 036 186.8
Jul. 29, 2008 (EP) PCT/EP2008/006220

(51) Int. Cl. A61K 8/55; C07F 9/09; A61Q 15/00; A61Q 19/00

(52) U.S. Cl. 424/66; 558/186; 514/129; 424/68; 424/616

(57) ABSTRACT

The invention relates to phosphoric acid esters containing A) at least one structural unit derived from substances of constituent a), the substances of constituent a) being selected from orthophosphoric acid and at least one of the derivatives thereof; B) at least one structural unit derived from substances of constituent b), the substances of constituent b) being selected from at least one compound of formula (I) R²—O—(CH₂CH₂O)_{U}(C₃H₆O)_{V}(DO)_{w}—H; and C) at least one structural unit derived from substances of constituent c), the substances of constituent c) being selected from at least one polyol which has more than 2 OH groups and can also carry at least one alkoxylate group. The alkoxylate groups are respectively formed from at least one unit selected from CH₂CH₂O, C₃H₆O and C₄H₈O units that can respectively be arranged in a block or in a statistically distributed manner inside the alkoxylate groups. The phosphoric acid esters contain at least two phosphorus atoms per molecule, said phosphorus atoms being bridged by a structural unit derived from the polyols having more than two OH groups or from the polyols having more than two OH groups and carrying at least one of the alkoxylate groups. The phosphoric acid esters according to the invention can be used advantageously for producing cosmetic, pharmaceutical and dermatological compositions.

This invention relates to esters formed from phosphoric acid or phosphoric acid derivatives, fatty alcohol, which is optionally alkoxylated, and polyol, and also to their use as associative thickeners, particularly in cosmetic, pharmaceutical or dermatological compositions.

Cosmetic products have to meet high requirements. They shall have a clear appearance, be generally recognized as safe by toxicologists and ecotoxicologists, create a pleasant skin feel and have excellent rheological behavior which is constant over a wide pH range.

Water- or solvent-containing multicomponent systems such as emulsions or suspensions are frequently adjusted to higher viscosities, i.e., thickened, for economic reasons, for performance reasons or for stability reasons.

For instance, increasing the viscosity of the external or internal phase of emulsions or suspensions lengthens the time to separation of the components of such a system distinctly, which manifests itself in a lengthening of the storage time. Increasing the viscosity also improves for many products their uniform distributability on nonplanar surfaces in particular.

The more uniform distribution and lengthened active time enhances the efficacy. In addition to the performance advantages mentioned, the high viscosity of such products also offers further advantages in relation to manufacture, packaging, filling and storage and also in transportation.

The technical literature contains reports of a large number of different systems for adjusting the rheological properties of aqueous or solvent-containing systems, emulsions or suspensions. Known examples are cellulose ethers and other cellulose derivatives (for example carboxymethylcellulose, hydroxyethylcellulose), gelatin, starch and starch derivatives, sodium alginates, fatty acid polyethylene glycol esters, agar, tragacanth or dextrins. By way of synthetic polymers, various materials are used, examples being polyvinyl alcohols, polyacrylamides, polyacrylic acid and various salts of polyacrylic acid, polyvinylpyrrolidone, polyvinyl methyl ether, polyethylene oxides, copolymers of maleic anhydride and vinyl methyl ether, and also diverse mixtures and copolymers thereof.

However, the compounds mentioned display manifold disadvantages in use. For instance, cellulose derivatives and, in general, materials based on natural raw materials and the formulations resulting therefrom are very vulnerable to bacteria. Technically, they usually form unpleasant, “ropey” gels.

Fatty acid polyethylene glycol esters tend to hydrolyze in the presence of water and the resulting insoluble fatty acids cause undesirable clouding. Thickeners of natural origin (for example agar or tragacanth) fluctuate substantially in composition, depending on provenience.

U.S. Pat. No. 5,129,462 describes shampoo formulations comprising polyethylene glycol polyol fatty acid esters, particularly polyethelyne glycol pentaerythritol fatty acid esters as thickeners. The processing and formulatability of this class of compounds is impaired by their high melting points or setting points.

EP 1 518 900 and EP 1 344 518 disclose cosmetic and pharmaceutical preparations comprising oxyalkylated polyglycerol esters as thickeners, dispersants for aqueous, aqueous-alcoholic and aqueous-surfactant preparations and as emulsifiers; suspending agents having a thickening effect and consistency regulators for emulsions and suspensions.

The associative thickeners described in U.S. Pat. No. 5,129,462, EP 1 518 900 and EP 1 344 518 still have room for improvement with regard to their thickening performance, specifically in purely aqueous systems, where they only form cloudy gels, but also with regard to their stability at low pH. At below pH 5 their gels and thickened surfactant solutions are not stable in storage, but very rapidly lose viscosity.

It is an object of the present invention to provide a novel class of substances which is suitable for use in cosmetic products, gives a clear appearance in the formulations and even in a very acidic medium engenders a high thickening capacity in the event of thermal stress and long storage times, and combines these properties with excellent thickening performance.

We have found that this object is achieved, surprisingly, by esters of phosphoric acid or esters of phosphoric acid derivatives with optionally alkoxylated fatty alcohols, the esters being characterized in that at least 2 phosphorus atoms are bridged via groups derived from polyols having more than 2 OH groups or the corresponding alkoxylated polyols.

The present invention accordingly provides phosphoric esters comprising
A) one or more structural units derived from substances of component a), the substances of component a) being selected from orthophosphoric acid and one or more of its derivatives and the one or more derivatives of orthophosphoric acid preferably being selected from polyphosphoric acid, tetraphosphorus decaoxide, phosphoryl chloride and phosphorus pentachloride,
B) one or more structural units derived from substances of component b), the substances of component b) being selected from one or more compounds of formula (I)

R²—O—(CH₂CH₂O)ᵤ(C₃H₆O)ᵥ(DO)_{w}—H (I)
where
R² is a linear or branched, saturated alkyl group having 6 to 30, preferably 8 to 22 and more preferably 12 to 18 carbon atoms, or is a linear or branched mono- or polyunsaturated alkenyl group having 6 to 30, preferably 8 to 22 and more preferably 12 to 18 carbon atoms,
D is a linear or branched saturated alkylene group having 4 to 20 carbon atoms, is a linear or branched mono- or polyunsaturated alkenylene group having 4 to 20 carbon atoms or is —CH(phenyl)CH₂—,
u is a number from 0 to 200, preferably from 2 to 150, more preferably from 5 to 100 and even more preferably from 10 to 50,
v is a number from 0 to 100, preferably from 0 to 50 and more preferably from 0 to 20,
w is a number from 0 to 100, preferably from 0 to 50 and more preferably from 0 to 20, and
where the groups CH₂CH₂O, C₃H₆O and DO from the compounds of formula (I) can be arranged blocklike or randomly distributed, and
C) one or more structural units derived from substances of component c), the substances of component c) being selected from one or more polyols having more than 2 OH groups which may also bear one or more alkoxylate groups and where the alkoxylate groups are each constructed of one or more units selected from CH₂CH₂O—, C₃H₆O— and C₄H₈O— units which may each be arranged blocklike or randomly distributed within the alkoxylate groups,
   and wherein the phosphoric esters contain at least 2 phosphorus atoms per molecule which are bridged via a structural unit derived from the polyols having more than 2 OH groups or derived from the polyols having more than 2 OH groups which bear one or more of the alkoxylate groups,

The phosphoric esters of the present invention do not contain any oxygen-oxygen bond —O—O—. The structural units derived from the substances of components a), b) and c) are bonded to each other via one oxygen atom —O— only.

In one preferred embodiment of the present invention, the phosphoric esters of the present invention are characterized in that the substances of component c) are selected from glycerol, diglycerol, polyglycerol, pentaerythritol, dipentaerythritol, pentaerythritol oligomers, trimethylolpropane, threitol, erythritol, adonitol, arabitol, xylitol, mannitol, sorbitol, inositol, glucose, mannose, fructose, sorbose, arabinose, xylose, ribose, mannopyranose, galactopyranose, glucopyranose, maltose, sucrose, amino sugar, ascorbic acid, glucamides and gluconamides, which may also bear one or more alkoxylate groups and where the alkoxylate groups are each constructed of one or more units selected from CH₂CH₂O—, C₃H₆O— and C₄H₈O— units which each may be arranged blocklike or randomly distributed within the alkoxylate groups.

In a particularly preferred embodiment of the present invention, the phosphoric esters of the present invention are characterized in that the substances of component c) are selected from pentaerythritol, glycerol and diglycerol, preferably pentaerythritol, which may also bear one or more alkoxylate groups and where the alkoxylate groups are each constructed of one or more units selected from CH₂CH₂O—, C₃H₆O— and C₄H₈O— units which each may be arranged blocklike or randomly distributed within the alkoxylate groups.

In a further preferred embodiment of the present invention, the phosphoric esters of the present invention are characterized in that the substances of component c) bear one or more alkoxylate groups. Preference among these phosphoric esters of the present invention is given to those wherein the alkoxylate groups of the substances of component c) consist of CH₂CH₂O— groups and the number of CH₂CH₂O— groups per polyol molecule having more than 2 OH groups is in the range from 1 to 150, preferably in the range from 5 to 130 and more preferably in the range from 10 to 110.

In a further preferred embodiment of the present invention, the phosphoric esters of the present invention are characterized in that the substances of component b) are selected from one or more compounds of formula (II),

R²—O—(CH₂CH₂O)ᵤ₁(C₃H₆O)ᵥ₁—H (II)

where

R² is a linear or branched saturated alkyl group having 6 to 30, preferably 8 to 22 and more preferably 12 to 18 carbon atoms, or is a linear or branched mono- or polyunsaturated alkenyl group having 6 to 30, preferably 8 to 22 and more preferably 12 to 18 carbon atoms,

u1 is a number from 1 to 200, preferably from 2 to 150, more preferably from 5 to 100 and even more preferably from 10 to 50, and

v1 is a number from 1 to 100, preferably from 1 to 50 and more preferably from 1 to 20,

and wherein the CH₂CH₂O— and C₃H₆O— units may be arranged blocklike or randomly distributed.

In a further preferred embodiment of the present invention, the phosphoric esters of the present invention are characterized in that the substances of component b) are selected from one or more compounds of formula (III),

R²—O—(CH₂CH₂O)ᵤ₁—H (III)

where

R² is a linear or branched saturated alkyl group having 6 to 30, preferably 8 to 22 and more preferably 12 to 18 carbon atoms, or is a linear or branched mono- orpolyunsaturated alkenyl group having 6 to 30, preferably 8 to 22 and more preferably 12 to 18 carbon atoms, and

u1 is a number from 1 to 200, preferably from 2 to 150, more preferably from 5 to 100 and even more preferably from 10 to 50.

In a further preferred embodiment of the present invention, the phosphoric esters of the present invention are characterized in that the one or more structural units derived from the one or more compounds of formula (I) are structural units wherein u is a number from 1 to 200, preferably from 2 to 150, more preferably from 5 to 100 and even more preferably from 10 to 50, v and w are 0 and the radical R²—O— is derived from alcohols selected from octanol, decanol, dodecanol, tetradecanol, hexadecanol, octadecanol, eicosanol, behenyl alcohol, fatty alcohols having C-chain cuts between 8 and 22, preferably C₁₀/C₁₂ fatty alcohol, C₁₂/C₁₄ fatty alcohol, C₁₂/C₁₅ fatty alcohol and C₁₆/C₁₈ fatty alcohol, branched fatty alcohols, preferably Guerbet alcohols and monounsaturated fatty alcohols, preferably delta-9-cis-hexadecanol, delta-9-cis-octadecanol, trans-9-octadecanol and cis-delta-11-octadecanol.

In a particularly preferred embodiment of the present invention, the phosphoric esters of the present invention are characterized in that the one or more structural units derived from the one or more compounds of formula (I) are structural units derived from C_{16/18} fatty alcohol ethoxylates with 10-50 ethylene oxide units, preferably derived from substances selected from C_{16/18} fatty alcohol ethoxylate with 11 ethylene oxide units, C_{16/18} fatty alcohol ethoxylate with 25 ethylene oxide units and C_{16/18} fatty alcohol ethoxylate with 50 ethylene oxide units.

In a further preferred embodiment of the present invention, the phosphoric esters of the present invention are characterized in that the total number in the phosphoric esters of ethylene oxide units in the structural units derived from the substances of component b) and the substances of component c) is together in the range from 30 to 100 and preferably in the range from 40 to 80 per fatty alcohol end group emerged from the compounds of formula (I).

In a further particularly preferred embodiment of the present invention, the phosphoric esters of the present invention are characterized in that they are obtainable from the reaction of
a) 5 to 10 mol of a C₁₂-C₂₂ fatty alcohol ethoxylate, preferably of a C_{16/18} fatty alcohol ethoxylate having 10-50 ethylene oxide units and preferably having 11 or 25 ethylene oxide units,
b) 1 mol of a polyol selected from pentaerythritol, glycerol and diglycerol each ethoxylated with 50 to 150 ethylene oxide units, and
c) 2 to 5 mol of orthophosphoric acid or one or more of its derivatives, in which case the one or more derivatives of orthophosphoric acid are preferably selected from polyphosphoric acid, tetraphosphorus decaoxide, phosphoryl chloride and phosphorus pentachloride.

In an especially preferred embodiment of the present invention, the phosphoric esters of the present invention are characterized in that they are obtainable from the reaction of
a) 6 to 10 mol, preferably 8 mol, of a C₁₂-C₂₂ fatty alcohol ethoxylate, preferably of a C_{16/18} fatty alcohol ethoxylate having 10-50 ethylene oxide units and preferably having 11 or 25 ethylene oxide units,
b) 1 mol of pentaerythritol ethoxylated with 50 to 150 and preferably 100 ethylene oxide units, and
c) 3 to 5 mol, preferably 4 mol, of orthophosphoric acid or one or more of its derivatives, in which case the one or more derivatives of orthophosphoric acid are preferably selected from polyphosphoric acid, tetraphosphorus decaoxide, phosphoryl chloride and phosphorus pentachloride.

Of the just-mentioned phosphoric esters of the present invention, particular preference is given in turn to those which are obtainable from the reaction of
a) 8 mol of a C₁₂-C₂₂ fatty alcohol ethoxylate, preferably of a C_{16/18} fatty alcohol ethoxylate, having 10-50 ethylene oxide units and preferably having 11 or 25 ethylene oxide units,
b) 1 mol of pentaerythritol ethoxylated with 100 ethylene oxide units, and
c) 4 mol of orthophosphoric acid.

In a further especially preferred embodiment of the present invention, the phosphoric esters of the present invention are characterized in that they are obtainable from the reaction of
a) 6 mol of a C₁₂-C₂₂ fatty alcohol ethoxylate, preferably of a C_{16/18} fatty alcohol ethoxylate, having 10-50 ethylene oxide units and preferably having 11 or 25 ethylene oxide units,
b) 1 mol of glycerol ethoxylated with 100 ethylene oxide units, and
c) 3 mol of orthophosphoric acid.

In a further especially preferred embodiment of the present invention, the phosphoric esters of the present invention are characterized in that they are obtainable from the reaction of
a) 8 mol of a C₁₂-C₂₂ fatty alcohol ethoxylate, preferably of a C_{16/18} fatty alcohol ethoxylate, having 10-50 ethylene oxide units and preferably having 11 or 25 ethylene oxide units,
b) 1 mol of diglycerol ethoxylated with 100 ethylene oxide units, and
c) 4 mol of orthophosphoric acid.

In a further preferred embodiment of the present invention, the phosphoric esters of the present invention are characterized in that at least 75%, preferably from 80 to 100% and more preferably from 85 to 100% of the maximum number of the esterifiable functions theoretically obtainable from the substances of component a) in the phosphoric esters are in an esterified state.

The remaining free valences on the phosphorus atom can be acid groups, but also counter ions selected from Li⁺, Na⁺, K⁺, Mg⁺⁺, Ca⁺⁺, Al⁺⁺⁺, NH₄⁺ and quaternary ammonium ions [HNR^{a}R^{b}R^{c}]⁺, where R^{a}, R^{b} and R^{c} independently can be hydrogen, a linear or branched alkyl group having 1 to 22 carbon atoms, a linear or branched mono- or polyunsaturated alkenyl group having 2 to 22 carbon atoms, a linear monohydroxyalkyl group having 2 to 10 carbon atoms, preferably a monohydroxyethyl or monohydroxypropyl group, and also a linear or branched dihydroxyalkyl group having 3 to 10 carbon atoms.

The degree of neutralization of the unsubstituted phosphorus valences (P-OH) can be between 0% and 100%. In one preferred embodiment of the present invention, the degree of neutralization is from 0-20%, In another preferred embodiment of the invention, the degree of neutralization is from 20.1-100%.

In a further preferred embodiment of the present invention, the phosphoric esters of the present invention are characterized in that, in addition to the structural units derived from substances of components (a), (b) and (c), they additionally comprise

D) one or more structural units derived from substances of component d), the substances of component d) being selected from one or more diols of formula (IV)

HO—(CH₂CH₂O)ₐ(C₃H₆O)_{b}(DO)_{c}—H (IV)

where

D is as defined in formula (I),

a is a number from 0 to 800, preferably from 0 to 250, more preferably from 10 to 200 and even more preferably from 20 to 100,

b is a number from 0 to 100 and preferably from 0 to 50,

c is a number from 0 to 100 and preferably from 0 to 50,

where the sum total a+b+c is ≧1, preferably from 25 to 250, and the groups CH₂CH₂O, C₃H₆O and DO from the compounds of formula (II) can be arranged blocklike or randomly distributed.

Among the phosphoric esters of the present invention just mentioned, preference is given in turn to those which contain structural units derived from substances of component d), the substances of component d) being selected from ethylene glycol, diethylene glycol, triethylene glycol, tetraethylene glycol, polyethylene glycol (PEG) having molecular weights from 200 to 35 000, preferably PEG 200, PEG 300, PEG 400, PEG 600, PEG 800, PEG 1000, PEG 1500, PEG 2000, PEG 3000, PEG 3350, PEG 4000, PEG 6000, PEG 8000, propylene glycol, dipropylene glycol, tripropylene glycol, polypropylene glycol, polybutylene glycol, copolymers of ethylene oxide and propylene oxide having molecular weights of 200 to 35 000, 1,2-butanediol, 1,3-butanediol, 1,4-butanediol, 1,2-pentanediol, 1,3-pentanediol, 1,4-pentanediol, 1,5-pentanediol, 1,2-hexanediol, 1,3-hexanediol, 1,4-hexanediol, 1,5-hexanediol, 1,6-hexanediol and 1,12-dodecanediol.

The present invention also further provides mixtures comprising one or more phosphoric esters of the present invention. In one preferred embodiment of the present invention, these mixtures may also comprise phosphoric esters having just one phosphorus atom per molecule, in particular those of the formula

[R²—O—(CH₂CH₂O)ᵤ(C₃H₆O)ᵥ(DO)_{w}—]₃P═O,

where R², u, v, w and D are each as defined above under the compounds of formula (I). Among these mixtures, preference is in turn given to those which consist of the phosphoric esters mentioned. The proportion of the mixtures of the present invention which is attributable to the phosphoric esters of the present invention is preferably greater than 50% by weight, more preferably in the range from 70% to 100% by weight and even more preferably in the range from 80% to 100% by weight. In another preferred embodiment of the present invention, the mixtures of the present invention consist of the phosphoric esters of the present invention.

The present invention also provides a process for preparing the phosphoric esters of the present invention.

The phosphoric esters of the present invention can be prepared by reacting phosphoric acid or derivatives thereof with alcohol, preferably fatty alcohol ethoxylate, and polyol having more than 2 OH groups or a corresponding polyol containing alkoxylate groups, and, if appropriate, diol, at temperatures of 150 to 250° C., preferably of 180 to 240° C. and more preferably of 200 to 230° C., preferably without addition of a catalyst.

The present invention therefore further provides a process for preparing a phosphoric ester, which comprises reacting a phosphoric acid component selected from orthophosphoric acid and one of its derivatives with an alcohol component, preferably fatty alcohol ethoxylate, and polyol having more than 2 OH groups or a corresponding polyol comprising alkoxylate groups at temperatures of 150 to 250° C., preferably of 180 to 240° C. and more preferably of 200 to 230° C., preferably without addition of a catalyst.

Suitable phosphoric acid derivatives are polyphosphoric acid, tetraphosphorus decaoxide, phosphoryl chloride and phosphorus pentachloride.

In a preferred embodiment of the process of the present invention, a substance selected from orthophosphoric acid, polyphosphoric acid and tetraphosphorus decaoxide, preferably orthophosphoric acid, is reacted as phosphoric acid component.

The esterification is preferably carried on such that essentially neutral phosphoric esters of the present invention are present. Preference is given to a degree of conversion >75%, i.e., more than 75% of all esterifiable functions of the phosphoric acid or phosphoric acid derivatives are esterified. A degree of conversion >80% is particularly preferred and >85% even more particularly preferred.

The remaining free valences on the phosphorus atom can be acid groups, but also counter ions selected from Li⁺, Na⁺, K⁺, Mg⁺⁺, Ca⁺⁺, Al⁺⁺⁺, NH₄⁺ and quaternary ammonium ions [HNR¹R²R³]⁺, where R¹, R² and R³ independently can be hydrogen, a linear or branched alkyl group having 1 to 22 carbon atoms, a linear or branched mono- or polyunsaturated alkenyl group having 2 to 22 carbon atoms, a linear monohydroxyalkyl group having 2 to 10 carbon atoms, preferably a monohydroxyethyl or monohydroxypropyl group, and also a linear or branched dihydroxyalkyl group having 3 to 10 carbon atoms.

The degree of neutralization of the unsubstituted phosphorus valences (P—OH) can be between 0% and 100%. In one preferred embodiment of the present invention, the degree of neutralization is from 0-20%. In another preferred embodiment of the invention, the degree of neutralization is from 20.1%-100%.

The phosphoric esters of the present invention have excellent thickening capacity not only for compositions on an aqueous or aqueous-alcoholic basis but also for compositions on an aqueous-surfactant basis and tolerate even organic solvents such as alcohols.

The phosphoric esters of the present invention are further very useful in the manufacture of cosmetic, pharmaceutical and dermatological compositions.

The present invention therefore further provides a cosmetic, pharmaceutical or dermatological composition, characterized in that it comprises one or more phosphoric esters of the present invention.

The phosphoric esters of the present invention have manifold possible uses and are suitable for use in aqueous, aqueous-alcoholic and aqueous-surfactant compositions, emulsions, suspensions, dispersions, powders and sprays.

In a preferred embodiment of the present invention, the compositions of the present invention are therefore present in the form of aqueous, aqueous-alcoholic or aqueous-surfactant compositions, emulsions, suspensions, dispersions, powders or sprays.

It is particularly advantageous that the thickening capacity of the phosphoric esters of the present invention is also marked in a strong acidic medium.

The phosphoric esters of the present invention are therefore particularly useful for thickening and stabilizing acidic cosmetic compositions. These can be for example cosmetic compositions comprising hydroxyacids, such as lactic acid, glycolic acid, salicylic acid, citric acid or polyglycol diacids in free or partial neutralization. It is further possible to stabilize formulations comprising vitamin C or vitamin C derivatives, dihydroxyacetone or skin-whitening actives such as arbutin or glycyrrhetic acid and salts thereof.

In a further preferred embodiment of the present invention, the compositions of the present invention have a pH in the range from 2 to 10, preferably in the range from 2 to 6, more preferably in the range from 2.5 to 5 and even more preferably in the range from 3 to 4.5.

The phosphoric esters of the present invention are also very useful as thickeners of electrolyte-containing compositions.

The electrolytes used are inorganic salts, preferably ammonium or metal salts, more preferably of halides, for example CaCl₂, MgCl₂, LiCl, KCl and NaCl, carbonates, bicarbonates, phosphates, sulfates, nitrates, more preferably sodium chloride, and/or organic salts, preferably ammonium or metal salts, more preferably of glycolic acid, lactic acid, citric acid, tartaric acid, mandelic acid, salicylic acid, ascorbic acid, pyruvic acid, fumaric acid, retinoic acid, sulfonic acids, benzoic acid, kojic acid, fruit acid, malic acid, gluconic acid and galacturonic acid.

As electrolyte, the compositions of the present invention may also comprise mixtures of various salts.

In a further preferred embodiment of the present invention, the compositions of the present invention comprise one or more electrolytes.

These include aqueous antiperspirant formulations comprising aluminum salts, preferably aluminum chlorohydrate or aluminum-zirconium complex salts.

The content of the one or more electrolytes is, based on the entire composition of the present invention, preferably in the range from 0.1% to 20.0% by weight, more preferably in the range from 0.2% to 10.0% by weight and even more preferably in the range from 0.5% to 5.0% by weight.

It is furthermore very advantageous that the phosphoric esters of the present invention both thicken and stabilize compositions comprising oxidizing agents, preferably hydrogen peroxide, for example hair colorants.

In a further preferred embodiment of the present invention, the compositions of the present invention comprise hydrogen peroxide or hydrogen peroxide releasers and are preferably present in the form of gels or creams.

Useful hydrogen peroxide releasers preferably include inorganic peracids, preferably peroxosulfuric acid, peroxodisulfuric acid, peroxocarbonates, and also organic peracids, preferably peracetic acid.

In a further preferred embodiment of the present invention, the compositions of the present invention are acidic hydrogen peroxide bleaching gels or creams.

The phosphoric esters of the present invention are particularly useful for thickening cosmetic, pharmaceutical and dermatological compositions comprising one or more surfactants. It is preferably shampoos and shower baths which are concerned here.

In a further preferred embodiment of the present invention, the compositions of the present invention comprise one or more surfactants.

In a further preferred embodiment of the present invention, the phosphoric esters of the present invention are used in rinse-off products, preferably shampoos, shower baths, shower gels and foam baths.

The phosphoric esters of the present invention are very useful as thickener, consistency regulator, emulsifier, sensory additive, solubilizer, dispersant, suspension medium, glidant, adhesive and stabilizer.

The present invention therefore also provides for the use of one or more of the phosphoric esters of the present invention as thickener, consistency regulator, emulsifier, sensory additive, solubilizer, dispersant, suspension medium, glidant, adhesive or stabilizer, preferably the use as thickener.

In a preferred embodiment of the present invention, phosphoric esters of the present invention are used for thickening cosmetic, pharmaceutical and dermatological compositions comprising one or more surfactants, preferably for thickening shampoos or shower baths.

The phosphoric esters of the present invention can be used as thickeners for compositions on an aqueous or aqueous-alcoholic basis, for example hair gels, moisturizing gels, antiperspirant gels, bleaching gels, conditioners and disinfection gels. The phosphoric esters of the present invention are further useful as stabilizer, dispersant and consistency regulator for aqueous-surfactant preparations, for example shampoos, shower baths, shower gels and foam baths and for improving skin mildness and skin compatibility.

The thickening effect of the phosphoric esters of the present invention in aqueous-surfactant compositions is brought about by the association of the hydrophobic end groups with the surfactant micelles, and can be controlled through the choice of the ethoxylate end groups of the phosphoric esters of the present invention and through the choice of the surfactants.

The suspending/dispersing and stabilizing effect of the phosphoric esters of the present invention in aqueous-surfactant compositions is due to the association of the hydrophobic end groups and of the liquid components, for example oils and silicone oils, that are insoluble in aqueous-surfactant compositions, or of the insoluble solids components, for example pigments and active ingredients such as zinc pyrithiones.

The phosphoric esters of the present invention are similarly useful as thickeners and dispersants, as emulsifiers, suspending agents having a thickening effect and consistency regulators for emulsions and suspensions, such as conditioners, and also as glidant, adhesive, thickener, dispersing and emulsifying agents of decorative, solids-containing preparations. Mixtures of the phosphoric esters of the present invention can also be used. The emulsifying, stabilizing and/or consistency-regulating effect of the phosphoric esters of the present invention in emulsions is caused and enhanced, respectively, by an association between the hydrophobic end groups and also by an interaction of the hydrophobic end groups with the hydrophobic oil components.

In deodorant or antiperspirant formulations comprising aluminum salts, preferably aluminum chlorohydrate or aluminum-zirconium complex salts, the phosphoric esters of the present invention that are included therein show the advantage that they reduce the formation of white residues on clothing donned after application of the formulations to the skin.

The present invention therefore also provides for the use of one or more phosphoric esters of the present invention in deodorant or antiperspirant formulations, in particular in deodorant or antiperspirant formulations comprising aluminum salts, preferably aluminum chlorohydrate or aluminum-zirconium complex salts, for reducing the formation of white residues on the clothing after using the deodorant or antiperspirant formulations on the skin.

In one further preferred embodiment of the present invention, the cosmetic, pharmaceutical or dermatological compositions of the present invention are present as emulsions.

The emulsions can be not only water-in-oil emulsions but also oil-in-water emulsions, microemulsions and multiple emulsions.

The emulsions can be prepared in a known manner, i.e., for example, by hot, hot/cold or PIT emulsification.

The nonaqueous portion of the emulsions, which is largely made up of the emulsifier, the thickener and the oil body, is typically in the range from 5% to 95% by weight, preferably in the range from 15% to 75% by weight. It follows that the emulsions can comprise 5% to 95% by weight and preferably 25% to 85% by weight of water, depending on whether lotions having a comparatively low viscosity or creams and ointments of high viscosity are to be produced.

In a further preferred embodiment of the present invention, the phosphoric esters of the present invention are used in leave-on products, preferably skincare agents such as day creams, night creams, moisturizing lotions and gels, aqueous gels, for example facial toners, care creams, nutrient creams, body lotions, ointments, sunscreen compositions, lip care compositions, antiperspirants and deodorants.

They are further also useful for surfactant-free aqueous compositions and emulsions and also for hair treatments, hair rinses and hair gels, but also for permanent wave compositions, hair colorants, and also for decorative cosmetics, for example make-ups, eye shadows, lipsticks, mascara and the like.

The compositions of the present invention comprise, based on the final cosmetic, pharmaceutical or dermatological compositions, preferably from 0.01% to 10.0% by weight, more preferably from 0.1% to 6.0% by weight and even more preferably from 0.5% to 3.0% by weight of the phosphoric esters of the present invention.

The compositions of the present invention may comprise anionic, cationic, nonionic, ampholytic surfactants and/or betaine surfactants.

The total amount of the surfactants used in the compositions of the present invention (in the case of rinse-off products for example) is, based on the final compositions of the present invention, preferably in the range from 1.0% to 70.0% by weight, more preferably in the range from 5.0% to 40.0% by weight and even more preferably in the range from 10.0% to 35.0% by weight.

The anionic surfactants are preferably (C₁₀-C₂₂)-alkyl and alkylene carboxylates, alkyl ether carboxylates, fatty alcohol sulfates, fatty alcohol ether sulfates, alkylamide sulfates and sulfonates, fatty acid alkylamide polyglycol ether sulfates, alkanesulfonates and hydroxyalkanesulfonates, olefinsulfonates, acyl esters of isethionates, α-sulfo fatty acid esters, alkylbenzenesulfonates, alkylphenol glycol ether sulfonates, sulfosuccinates, sulfosuccinic acid half-esters and diesters, fatty alcohol phosphates, fatty alcohol ether phosphates, protein-fatty acid condensation products, alkyl monoglyceride sulfates and sulfonates, alkyl glyceride ether sulfonates, fatty acid methyl taurides, fatty acid sarcosinates, sulforicinoleates, acyl glutamates and acyl glycinates. These compounds and mixtures thereof are used in the form of their water-soluble or water-dispersible salts, for example the sodium, potassium, magnesium, ammonium, mono-, di- and triethanolammonium, and analogous alkylammonium salts.

The amount of anionic surfactants in the compositions according to the invention is preferably from 2.0 to 30.0% by weight, particularly preferably from 5.0 to 25.0% by weight and especially preferably from 12.0 to 22.0% by weight, based on the final compositions.

Preferred cationic surfactants are quaternary ammonium salts, such as di(C₈-C₂₂)-alkyldimethylammonium chloride or bromide, preferably di(C₈-C₂₂)-alkyldimethylammonium chloride or bromide; (C₈-C₂₂)-alkyldimethylethylammonium chloride or bromide; (C₈-C₂₂)-alkyltrimethylammonium chloride or bromide, preferably cetyltrimethylammonium chloride or bromide and (C₈-C₂₂)-alkyltrimethylammonium chloride or bromide; (C₁₀-C₂₄)-alkyldimethylbenzylammonium chloride or bromide, preferably (C₁₂-C₁₈)-alkyldimethylbenzylammonium chloride, (C₈-C₂₂)-alkyldimethylhydroxyethylammonium chloride, phosphate, sulfate, lactate, (C₈-C₂₂)-alkylamidopropyltrimethylammonium chloride, methosulfate, N,N-bis(2-C₈-C₂₂-alkanoyloxyethyl)dimethylammonium chloride, methosulfate, N,N-bis(2-C₈-C₂₂-alkanoyloxyethyphydroxyethylmethylammonium chloride, methosulfate.

The amount of cationic surfactants in the compositions according to the invention is preferably 0.1 to 10.0% by weight, particularly preferably 0.5 to 7.0% by weight and especially preferably 1.0 to 5.0% by weight, based on the final compositions.

Preferred nonionic surfactants are fatty alcohol ethoxylates (alkylpolyethylene glycols); alkylphenol polyethylene glycols; fatty amine ethoxylates (alkylaminopolyethylene glycols); fatty acid ethoxylates (acyl polyethylene glycols); polypropylene glycol ethoxylates (Pluronics®); fatty acid alkanolamides (fatty acid amide polyethylene glycols); sucrose esters; sorbitol esters and sorbitan esters and polyglycol ethers thereof, and also C₈-C₂₂-alkyl polyglucosides.

The amount of nonionic surfactants in the compositions according to the invention (e.g. in the case of rinse-off products) is preferably in the range from 1.0 to 20.0% by weight, particularly preferably from 2.0 to 10.0% by weight and especially preferably from 3.0 to 7.0% by weight, based on the final compositions.

Furthermore, the compositions according to the invention can comprise amphoteric surfactants. These can be described as derivatives of long-chain secondary or tertiary amines which have an alkyl group with 8 to 18 carbon atoms and in which a further group is substituted by an anionic group which imparts the solubility in water, thus, for example, by a carboxyl, sulfate or sulfonate group. Preferred amphoteric surfactants are N—(C₁₂-C₁₈)-alkyl-β-aminopropionates and N—(C₁₂-C₁₈)-alkyl-β-iminodipropionates as alkali metal and mono-, di- and trialkylammonium salts; suitable further surfactants are also amine oxides. These are oxides of tertiary amines with a long-chain group having 8 to 18 carbon atoms and two mostly short-chain alkyl groups having 1 to 4 carbon atoms. Preference is given here, for example, to the C₁₀- to C₁₈-alkyldimethylamine oxides, fatty acid amidoalkyldimethylamine oxide.

A further preferred group of surfactants is betaine surfactants, also called zwitterionic surfactants. These contain in the same molecule a cationic group, in particular an ammonium group and an anionic group, which may be a carboxylate group, sulfate group or sulfonate group. Suitable betaines are preferably alkylbetaines such as cocobetaine or fatty acid alkylamidopropylbetaines, for example cocoacylamidopropyldimethylbetaine or the C₁₂- to C₁₈-dimethylaminohexanoates and/or the C₁₀- to C₁₈-acylamidopropanedimethylbetaines.

The amount of amphoteric surfactants and/or betaine surfactants in the compositions according to the invention is preferably from 0.5 to 20.0% by weight and particularly preferably from 1.0 to 10.0% by weight, based on the final compositions.

Preferred surfactants are laurel sulfate, laureth sulfate, cocoamidopropylbetaine, alkylbetaines such as cocobetaine, sodium cocoyl glutamate and lauroamphoacetate.

In a further preferred embodiment of the invention, the compositions according to the invention additionally also comprise, as foam-boosting agents, cosurfactants from the group of alkylbetaines, alkylamidobetaines, aminopropionates, aminoglycinates, imidazolinium betaines and sulfobetaines, amine oxides, fatty acid alkanolamides and polyhydroxyamides.

The compositions according to the invention can comprise, as further auxiliaries and additives, oil bodies, silicone oils, waxes, emulsifiers, coemulsifiers, solubilizers, stabilizers, cationic polymers, film formers, thickeners, gelling agents, superfatting agents, refatting agents, antimicrobial active ingredients, biogenic active ingredients, astringents, deodorizing agents, sun protection filters, antioxidants, humectants, solvents, dyes, fragrances, pearlizing agents, opacifiers and/or water-soluble silicones.

The oil bodies can advantageously be selected from the groups of triglycerides, natural and synthetic fatty substances, preferably esters of fatty acids with alcohols of low carbon number, e.g. with isopropanol, propylene glycol or glycerol, or esters of fatty alcohols with alkanoic acids of low carbon number or with fatty acids or from the group of alkyl benzoates, and also natural or synthetic hydrocarbon oils.

Triglycerides of linear or branched, saturated or unsaturated, optionally hydroxylated, C₈-C₃₀-fatty acids, in particular vegetable oils, such as sunflower oil, corn oil, soybean oil, rice oil, jojoba oil, babusscu oil, pumpkin oil, grapeseed oil, sesame oil, walnut oil, apricot oil, orange oil, wheatgerm oil, peach kernel oil, macadamia oil, avocado oil, sweet almond oil, lady's smock oil, castor oil, olive oil, peanut oil, rapeseed oil and coconut oil, and also synthetic triglyceride oils, e.g. the commercial product Myritol® 318, are suitable. Hydrogenated triglycerides are also preferred according to the present invention. Oils of animal origin, for example beef tallow, perhydrosqualene, lanolin, can also be used.

A further class of preferred oil bodies is the benzoic acid esters of linear or branched C₈₋₂₂-alkanols, e.g. the commercial products Finsolv® SB (isostearyl benzoate), Finsolv® TN (C₁₂-C₁₅-alkyl benzoate) and Finsolv® EB (ethylhexyl benzoate).

A further class of preferred oil bodies is the dialkyl ethers having in total 12 to 36 carbon atoms, in particular having 12 to 24 carbon atoms, such as, for example, di-n-octyl ether (Cetiol® OE), di-n-nonyl ether, di-n-decyl ether, di-n-undecyl ether, di-n-dodecyl ether, n-hexyl n-octyl ether, n-octyl n-decyl ether, n-decyl n-undecyl ether, n-undecyl n-dodecyl ether and n-hexyl n-undecyl ether, di-3-ethyldecyl ether, tert-butyl n-octyl ether, isopentyl n-octyl ether and 2-methylpentyl n-octyl ether, and di-tert-butyl ether and diisopentyl ether.

Branched saturated or unsaturated fatty alcohols having 6-30 carbon atoms, e.g. isostearyl alcohol, and Guerbet alcohols, are likewise suitable.

A further class of preferred oil bodies is hydroxycarboxylic acid alkyl esters. Preferred hydroxycarboxylic acid alkyl esters are full esters of glycolic acid, lactic acid, malic acid, tartaric acid or citric acid. Further esters of hydroxycarboxylic acids which are suitable in principle are esters of p-hydroxypropionic acid, of tartronic acid, of D-gluconic acid, sugar acid, mucic acid or glucuronic acid. Suitable alcohol components of these esters are primary, linear or branched aliphatic alcohols having 8 to 22 carbon atoms. Here, the esters of C₁₂-C₁₅-fatty alcohols are particularly preferred. Esters of this type are commercially available, e.g. under the trade name Cosmacol® from EniChem, Augusta Industriale.

A further class of preferred oil bodies is dicarboxylic acid esters of linear or branched C₂-C₁₀-alkanols, such as di-n-butyl adipate (Cetiol® B), di(2-ethylhexyl)adipate and di(2-ethylhexyl)succinate, and also diol esters, such as ethylene glycol dioleate, ethylene glycol diisotridecanoate, propylene glycol di(2-ethylhexanoate), propylene glycol diisostearate, propylene glycol dipelargonate, butanediol diisostearate and neopentyl glycol dicaprylate, and also diisotridecyl azelate.

Likewise preferred oil bodies are symmetrical, asymmetrical or cyclic esters of carbonic acid with fatty alcohols, glycerol carbonate or dicaprylyl carbonate (Cetiol® CC).

A further class of preferred oil bodies is the esters of dimers of unsaturated C₁₂-C₂₂-fatty acids (dimer fatty acids) with monovalent linear, branched or cyclic C₂-C₁₈-alkanols or with polyvalent linear or branched C₂-C₆-alkanols.

A further class of preferred oil bodies is hydrocarbon oils, for example those with linear or branched, saturated or unsaturated C₇-C₄₀-carbon chains, for example Vaseline, dodecane, isododecane, cholesterol, lanolin, synthetic hydrocarbons such as polyolefins, in particular polyisobutene, hydrogenated polyisobutene, polydecane, and hexadecane, isohexadecane, paraffin oils, isoparaffin oils, e.g. the commercial products of the Permethyl® series, squalane, squalene, and alicyclic hydrocarbons, e.g. the commercial product 1,3-di(2-ethylhexyl)cyclohexane (Cetiol® S), ozokerite, and ceresine.

Silicone oils and silicone waxes which are available are preferably dimethylpolysiloxanes and cyclomethicones, polydialkylsiloxanes R₃SiO(R₂SiO)ₓSiR₃, where R is methyl or ethyl, particularly preferably methyl, and x is a number from 2 to 500, for example the dimethicones available under the trade names VICASIL (General Electric Company), DOW CORNING 200, DOW CORNING 225, DOW CORNING 200 (Dow Corning Corporation), and also the dimethicones available under SilCare® Silicone 41M65, SilCare® Silicone 41M70, SilCare® Silicone 41M80 (Clariant GmbH), stearyldimethylpolysiloxane, C₂₀-C₂₄-alkyldimethylpolysiloxane, C₂₄-C₂₈-alkyldimethylpolysiloxane, but also the methicones available under SilCare® Silicone 41M40, SilCare® Silicone 41M50 (Clariant GmbH), furthermore trimethylsiloxysilicates [(CH₂)₃SiO)_{1/2}]ₓ[SiO₂]_{y}, where x is a number from 1 to 500 and y is a number from 1 to 500, dimethiconols R₃SiO[R₂SiO]ₓSiR₂OH and HOR₂SiO[R₂SiO]ₓSiR₂OH, where R is methyl or ethyl and x is a number up to 500, polyalkylarylsiloxanes, for example the polymethylphenylsiloxanes available under the trade names SF 1075 METHYLPHENYL FLUID (General Electric Company) and 556 COSMETIC GRADE PHENYL TRIMETHICONE FLUID (Dow Corning Corporation), polydiarylsiloxanes, silicone resins, cyclic silicones and amino-, fatty-acid-, alcohol-, polyether-, epoxy-, fluorine- and/or alkyl-modified silicone compounds, and also polyether siloxane copolymers.

The compositions according to the invention can comprise waxes, for example paraffin waxes, microwaxes and ozokerites, beeswax and its part fractions, and also beeswax derivatives, waxes from the group of homopolymeric polyethylenes or copolymers of α-olefins, and natural waxes such as rice wax, candelilla wax, carnauba wax, Japan wax or shellac wax.

Emulsifiers, coemulsifiers and solubilizers which can be used are nonionic, anionic, cationic or amphoteric surface-active compounds.

Suitable nonionogenic surface-active compounds are preferably: addition products of from 0 to 30 mol of ethylene oxide and/or 0 to 5 mol of propylene oxide onto linear fatty alcohols having 8 to 22 carbon atoms, onto fatty acids having 12 to 22 carbon atoms, onto alkylphenols having 8 to 15 carbon atoms in the alkyl group and onto sorbitan or sorbitol esters; (C₁₂-C₁₈)-fatty acid mono- and diesters of addition products of from 0 to 30 mol of ethylene oxide onto glycerol; glycerol mono- and diesters and sorbitan mono- and diesters of saturated and unsaturated fatty acids having 6 to 22 carbon atoms and optionally ethylene oxide addition products thereof; addition products of from 15 to 60 mol of ethylene oxide onto castor oil and/or hydrogenated castor oil; polyol and in particular polyglycerol esters, such as, for example, polyglycerol polyricinoleate and polyglycerol poly-12-hydroxystearate. Ethoxylated fatty amines, fatty acid amides, fatty acid alkanolamides and mixtures of compounds of two or more of these substance classes are likewise preferably suitable.

Suitable ionogenic coemulsifiers are, for example, anionic emulsifiers, such as mono-, di- or triphosphoric acid esters, soaps (e.g. sodium stearate), fatty alcohol sulfates, but also cationic emulsifiers such as mono-, di- and trialkyl quats and polymeric derivatives thereof.

Available amphoteric emulsifiers are preferably alkylaminoalkylcarboxylic acids, betaines, sulfobetaines and imidazoline derivatives.

Fatty alcohol ethoxylates selected from the group of ethoxylated stearyl alcohols, isostearyl alcohols, cetyl alcohols, isocetyl alcohols, oleyl alcohols, lauryl alcohols, isolauryl alcohols and cetylstearyl alcohols, in particular polyethylene glycol(13) stearyl ether, polyethylene glycol(14) stearyl ether, polyethylene glycol(15) stearyl ether, polyethylene glycol(16) stearyl ether, polyethylene glycol(17) stearyl ether, polyethylene glycol(18) stearyl ether, polyethylene glycol(19) stearyl ether, polyethylene glycol(20) stearyl ether, polyethylene glycol(12) isostearyl ether, polyethylene glycol(13) isostearyl ether, polyethylene glycol(14) isostearyl ether, polyethylene glycol(15) isostearyl ether, polyethylene glycol(16) isostearyl ether, polyethylene glycol(17) isostearyl ether, polyethylene glycol(18) isostearyl ether, polyethylene glycol(19) isostearyl ether, polyethylene glycol(20) isostearyl ether, polyethylene glycol(13) cetyl ether, polyethylene glycol(14) cetyl ether, polyethylene glycol(15) cetyl ether, polyethylene glycol(16) cetyl ether, polyethylene glycol(17) cetyl ether, polyethylene glycol(18) cetyl ether, polyethylene glycol(19) cetyl ether, polyethylene glycol(20) cetyl ether, polyethylene glycol(13) isocetyl ether, polyethylene glycol(14) isocetyl ether, polyethylene glycol(15) isocetyl ether, polyethylene glycol(16) isocetyl ether, polyethylene glycol(17) isocetyl ether, polyethylene glycol(18) isocetyl ether, polyethylene glycol(19) isocetyl ether, polyethylene glycol(20) isocetyl ether, polyethylene glycol(12) oleyl ether, polyethylene glycol(13) oleyl ether, polyethylene glycol(14) isocetyl ether, polyethylene glycol(15) isocetyl ether, polyethylene glycol(12) lauryl ether, polyethylene glycol(12) isolauryl ether, polyethylene glycol(13) cetylstearyl ether, polyethylene glycol(14) cetylstearyl ether, polyethylene glycol(15) cytylstearyl ether, polyethylene glycol(16) cytylstearyl ether, polyethylene glycol(17) cetylsteartyl ether, polyethylene glycol(18) cetylstearyl ether, polyethylene glycol(19) cetylstearyl ether are particularly preferably used.

Fatty acid ethoxylates selected from the group of ethoxylated stearates, isostearates and oleates, in particular polyethylene glycol(20) stearate, polyethylene glycol(21) stearate, polyethylene glycol(22) stearate, polyethylene glycol(23) stearate, polyethylene glycol(24) stearate, polyethylene glycol(25) stearate, polyethylene glycol(12) isostearate, polyethylene glycol(13), isostearate, polyethylene glycol(14) isostearate, polyethylene glycol(15) isostearate, polyethylene glycol(16) isostearate, polyethylene glycol(17) isostearate, polyethylene glycol(18) isostearate, polyethylene glycol(19) isostearate, polyethylene glycol(20) isostearate, polyethylene glycol(21) isostearate, polyethylene glycol(22) isostearate, polyethylene glycol(23) isostearate, polyethylene glycol(24) isostearate, polyethylene glycol(25) isostearate, polyethylene glycol(12) oleate, polyethylene glycol(13) oleate, polyethylene glycol(14) oleate, polyethylene glycol(15) oleate, polyethylene glycol(16) oleate, polyethylene glycol(17) oleate, polyethylene glycol(18) oleate, polyethylene glycol(19) oleate, polyethylene glycol(20)oleate are likewise preferred.

Sodium laureth-11 carboxylate can advantageously be used as ethoxylated alkylether carboxylic acid or salts thereof.

Ethoxylated triglycerides which can be used are advantageously polyethylene glycol(60) evening primrose glycerides.

It is furthermore advantageous to select the polyethylene glycol glycerol fatty acid esters from the group polyethylene glycol(20) glyceryl laurate, polyethylene glycol(6) glyceryl caprate/caprinate, polyethylene glycol(20) glyceryl oleate, polyethylene glycol(20) glyceryl isostearate and polyethylene glycol(18) glyceryl oleate/cocoate.

Among the sorbitan esters, polyethylene glycol(20) sorbitan monolaurate, polyethylene glycol(20) sorbitan monostearate, polyethylene glycol(20) sorbitan monoisostearate, polyethylene glycol(20) sorbitan monopalmitate, polyethylene glycol(20) sorbitan monooleate are particularly suitable.

Particularly advantageous coemulsifiers are glyceryl monostearate, glyceryl monooleate, diglyceryl monostearate, glyceryl isostearate, polyglyceryl-3 oleate, polyglyceryl-3 diisostearate, polyglyceryl-4 isostearate, polyglyceryl-2 dipolyhydroxystearate, polyglyceryl-4 dipolyhydroxystearate, PEG-30 dipolyhydroxystearate, diisostearoyl polyglyceryl-3 diisostearate, glycol distearate and polyglyceryl-3 dipolyhydroxystearate, sorbitan monoisostearate, sorbitan stearate, sorbitan oleate, sucrose distearate, lecithin, PEG-7-hydrogenated castor oil, cetyl alcohol, stearyl alcohol, behenyl alcohol, isobehenyl alcohol and polyethylene glycol(2) stearyl ether (steareth-2), alkylmethicone copolyols and alkyldimethicone copolyols, in particular cetyldimethicone copolyol, laurylmethicone copolyol.

The compositions according to the invention can comprise one or more of the emulsifiers, coemulsifiers or solubilizers in amounts of from 0.1 to 20.0% by weight, preferably 1.0 to 15.0% by weight and particularly preferably 3.0 to 10.0% by weight, based on the final compositions.

Stabilizers which can be used are metal salts of fatty acids, such as, for example, magnesium stearate, aluminum stearate and/or zinc stearate, preferably in amounts of from 0.1 to 10.0% by weight, preferably 0.5 to 8.0% by weight and particularly preferably 1.0 to 5.0% by weight, based on the final compositions.

Suitable cationic polymers are those known under the INCI name “Polyquaternium”, in particular Polyquaternium-31, Polyquaternium-16, Polyquaternium-24, Polyquaternium-7, Polyquaternium-22, Polyquaternium-39, Polyquaternium-28, Polyquaternium-2, Polyquaternium-10, Polyquaternium-11, and Polyquaternium 37&mineral oil&PPG trideceth (Salcare SC95), PVP-dimethylaminoethyl methacrylate copolymer, guar hydroxypropyltriammonium chlorides, and calcium alginate and ammonium alginate. Furthermore, cationic cellulose derivatives; cationic starch; copolymers of diallylammonium salts and acrylamides; quaternized vinylpyrrolidone/vinylimidazole polymers; condensation products of polyglycols and amines; quaternized collagen polypeptides; quaternized wheat polypeptides; polyethyleneimines; cationic silicone polymers, such as, for example, amidomethicones; copolymers of adipic acid and dimethylaminohydroxypropyldiethylenetriamine; polyaminopolyamide and cationic chitin derivatives, such as, for example, chitosan, can be used.

The compositions according to the invention can comprise one or more of the aforementioned cationic polymers in amounts of from 0.1 to 5.0% by weight, preferably 0.2 to 3.0% by weight and particularly preferably 0.5 to 2.0% by weight, based on the final compositions.

Furthermore, the compositions according to the invention can comprise film formers which, depending on the intended use, are selected from salts of phenylbenzimidazolesulfonic acid, water-soluble polyurethanes, for example C₁₀-polycarbamyl polyglyceryl ester, polyvinyl alcohol, polyvinyl pyrrolidone copolymers, for example vinyl pyrrolidone/vinyl acetate copolymer, water-soluble acrylic acid polymers/copolymers and esters or salts thereof, for example partial ester copolymers of acrylic acid/methacrylic acid, water-soluble cellulose, for example hydroxymethylcellulose, hydroxyethylcellulose, hydroxypropylcellulose, water-soluble quaterniums, polyquaterniums, carboxyvinyl polymers, such as carbomers and salts thereof, polysaccharides, for example polydextrose and glucan, vinyl acetate/crotonate, for example available under the trade name Aristoflex® A 60 (Clariant).

The compositions according to the invention can comprise one or more film formers in amounts of from 0.1 to 10.0% by weight, preferably from 0.2 to 5.0% by weight and particularly preferably from 0.5 to 3.0% by weight, based on the final compositions.

The desired viscosity of the compositions can be established by adding thickeners and gelling agents. Of suitability are preferably cellulose ethers and other cellulose derivatives (e.g. carboxymethylcellulose, hydroxyethylcellulose), gelatin, starch and starch derivatives, sodium alginates, fatty acid polyethylene glycol esters, agar, tragacanth or dextrin derivatives, in particular dextrin esters. Furthermore, metal salts of fatty acids, preferably having 12 to 22 carbon atoms, for example sodium stearate, sodium palmitate, sodium laurate, sodium arachidates, sodium behenate, potassium stearate, potassium palmitate, sodium myristate, aluminum monostearate, hydroxy fatty acids, for example 12-hydroxystearic acid, 16-hydroxyhexadecanoyl acid; fatty acid amides; fatty acid alkanolamides; dibenzalsorbitol and alcohol-soluble polyamides and polyacrylamides or mixtures of such are suitable. Furthermore, crosslinked and uncrosslinked polyacrylates such as carbomers, sodium polyacrylates or polymers containing sulfonic acid, such as ammonium acryloyldimethyltaurate/VP copolymer, can be used.

Preferably, the compositions according to the invention comprise 0.01 to 20.0% by weight, particularly preferably 0.1 to 10.0% by weight, especially preferably 0.2 to 3.0% by weight and very particularly preferably 0.4 to 2.0% by weight, of thickeners and/or gelling agents, based on the final compositions of the present invention.

Superfatting agents which can be used are preferably lanolin and lecithin, nonethoxylated and polyethoxylated or acylated lanolin derivatives and lecithin derivatives, polyol fatty acid esters, mono-, di- and triglycerides and/or fatty acid alkanolamides, where the latter simultaneously serve as foam stabilizers, which are preferably used in amounts of from 0.01 to 10.0% by weight, particularly preferably from 0.1 to 5.0% by weight and especially preferably from 0.5 to 3.0% by weight, based on the final compositions according to the invention.

The antimicrobial active ingredients used are cetyltrimethylammonium chloride, cetylpyridinium chloride, benzethonium chloride, diisobutylethoxyethyldimethylbenzylammonium chloride, sodium N-laurylsarcosinate, sodium N-palmethylsarcosinate, lauroylsarcosine, N-myristoylglycine, potassium N-laurylsarcosine, trimethylammonium chloride, sodium aluminum chlorohydroxylactate, triethyl citrate, tricetylmethylammonium chloride, 2,4,4′-trichloro-2′-hydroxydiphenyl ether (triclosan), phenoxyethanol, 1,5-pentanediol, 1,6-hexanediol, 3,4,4′-trichlorocarbanilide (triclocarban), diaminoalkylamide, for example L-lysine hexadecylamide, citrate heavy metal salts, salicylates, piroctoses, in particular zinc salts, pyrithiones and heavy metal salts thereof, in particular zinc pyrithione, zinc phenol sulfate, farnesol, ketoconazole, oxiconazole, bifonazole, butoconazole, cloconazole, clotrimazole, econazole, enilconazole, fenticonazole, isoconazole, miconazole, sulconazole, tioconazole, fluconazole, itraconazole, terconazole, naftifine and terbinafine, selenium disulfide and Octopirox®, iodopropynyl butylcarbamate, methylchloroisothiazolinone, methylisothiazolinone, methyldibromoglutaronitrile, AgCl, chloroxylenol, Na salt of diethylhexyl sulfosuccinate, sodium benzoate, and phenoxyethanol, benzyl alcohol, phenoxyisopropanol, parabens, preferably butyl, ethyl, methyl and propyl paraben, and Na salts thereof, pentanediol, 1,2-octanediol, 2-bromo-2-nitropropane-1,3-diol, ethylhexylglycerol, benzyl alcohol, sorbic acid, benzoic acid, lactic acid, imidazolidinylurea, diazolidinylurea, dimethyloldimethylhydantoin (DMDMH), Na salt of hydroxymethylglycinate, hydroxyethylglycine of sorbic acid and combinations of these active substances.

The compositions according to the invention comprise the antimicrobial active ingredients preferably in amounts of from 0.001 to 5.0% by weight, particularly preferably from 0.01 to 3.0% by weight and especially preferably from 0.1 to 2.0% by weight, based on the final compositions of the present invention.

The compositions according to the invention can furthermore comprise biogenic active ingredients selected from plant extracts, such as, for example, aloe vera, and also local anesthetics, antibiotics, antiphlogistics, antiallergics, corticosteroids, sebostatics, Bisabolol®, allantoin, Phytantriol®, proteins, vitamins selected from niacin, biotin, vitamin B2, vitamin B3, vitamin B6, vitamin B3 derivatives (salts, acids, esters, amides, alcohols), vitamin C and vitamin C derivatives (salts, acids, esters, amides, alcohols), preferably as sodium salt of the monophosphoric acid ester of ascorbic acid or as magnesium salt of the phosphoric acid ester of ascorbic acid, tocopherol and tocopherol acetate, and also vitamin E and/or derivatives thereof.

The compositions according to the invention can comprise biogenic active ingredients preferably in amounts of from 0.001 to 5.0% by weight, particularly preferably from 0.01 to 3.0% by weight and especially preferably from 0.1 to 2.0% by weight, based on the final compositions.

The compositions according to the invention can comprise astringents, preferably magnesium oxide, aluminum oxide, titanium dioxide, zirconium dioxide and zinc oxide, oxide hydrates, preferably aluminum oxide hydrate (boehmite) and hydroxides, preferably of calcium, magnesium, aluminum, titanium, zirconium or zinc, and also aluminum chlorohydrates, preferably in amounts of from 0 to 50% by weight, particularly preferably in amounts of from 0.01 to 10.0% by weight and especially preferably in amounts of from 0.1 to 10.0% by weight. Allantoin and bisabolol are preferred as deodorizing substances. These are preferably used in amounts of from 0.0001 to 10.0% by weight.

The compositions according to the invention can comprise microfine titanium dioxide, mica-titanium oxide, iron oxides, mica-iron oxide, zinc oxide, silicon oxides, ultramarine blue, chromium oxides as pigments/micropigments and also as sun protection filters.

The compositions according to the invention can comprise sun protection filters, preferably selected from 4-aminobenzoic acid, 3-(4′-trimethylammonium)benzylideneboran-2-one methyl sulfate, camphorbenzalkoniummethosulfate, 3,3,5-trimethylcyclohexyl salicylate, 2-hydroxy-4-methoxybenzophenone, 2-phenylbenzimidazole-5-sulfonic acid and its potassium, sodium and triethanolamine salts, 3,3′-(1,4-phenylenedimethine)bis(7,7-dimethyl-2-oxobicyclo[2.2.1]heptane-1-methanesulfonic acid) and its salts, 1-(4-tert-butylphenyl)-3-(4-methoxyphenyl)propane-1,3-dione, 3-(4′-sulfo)benzylidenebornan-2-one and its salts, 2-ethylhexyl 2-cyano-3,3-diphenylacrylate, polymers of N-[2(and 4)-(2-oxoborn-3-ylidenemethyl)benzyl]acrylamide, 2-ethylhexyl 4-methoxycinnamate, ethoxylated ethyl 4-aminobenzoate, isoamyl 4-methoxycinnamate, 2,4,6-tris[p-(2-ethylhexyloxycarbonyl)anilino]-1,3,5-triazine, 2-(2H-benzotriazol-2-yl)-4-methyl-6-(2-methyl-3-(1,3,3,3-tetramethyl-1-(trimethylsilyloxy)disiloxanyl)propyl)phenol, bis(2-ethylhexyl) 4,4′-[(6-[4-((1,1-dimethylethyl)aminocarbonyl)phenylamino]-1,3,5-triazin-2,4-yl)diimino]bisbenzoate, benzophenone-3, benzophenone-4 (acid), 3-(4′-methylbenzylidene)-D,L-camphor, 3-benzylidenecamphor, 2-ethylhexyl salicylate, 2-ethylhexyl 4-dimethylaminobenzoate, hydroxy-4-methoxybenzophenone-5-sulfonic acid (sulfisobenzone) and the sodium salt, 4-isopropylbenzyl salicylate, N,N,N-trimethyl-4-(2-oxoborn-3-ylidenemethyl)anilium methyl sulfate, homosalate (INN), oxybenzone (INN), 2-phenylbenzimidazole-5-sulfonic acid and its sodium, potassium and triethanolamine salts, octylmethoxycinnamic acid, isopentyl-4-methoxycinnamic acid, isoamyl-p-methoxycinnamic acid, 2,4,6-trianilino(p-carbo-2′-ethylhexyl-1′-oxy)-1,3,5-triazine (octyltriazone)phenol, 2-2(2H-benzotriazol-2-yl)-4-methyl-6-(2-methyl-3-(1,3,3,3-tetramethyl-1-(trimethylsilyl)oxy)disiloxanyl)propyl(drometrizoletrisiloxane)benzoic acid, 4,4-((6-(((1,1-dimethylethyl)amino)carbonyl)phenyl)amino)-1,3,5-triazine-2,4-diyl)diimino)bis,bis(2-ethylhexyl)ester)benzoic acid, 4,4-((6-(((1,1-dimethylethypamino)carbonyl)phenyl)amino)-1,3,5-triazine-2,4-diyl)diimino)bis,bis(2-ethylhexyl)ester), 3-(4′-methylbenzylidene)-D,L-camphor(4-methylbenzylidenecamphor), benzylidenecamphorsulfonic acid, octocrylene, polyacrylamidomethylbenzylidenecamphor, 2-ethylhexyl salicylate (octylsalicylate), ethyl-2-hexyl 4-dimethylaminobenzoate (octyldimethyl PABA), PEG-25 PABA, 2-hydroxy-4-methoxybenzophenone-5-sulfonic acid (benzophenone-5) and the Na salt, 2,2′-methylenebis-6-(2H-benzotriazol-2-yl)-4-(tetramethylbutyl)-1,1,3,3-phenol, sodium salt of 2-2′-bis(1,4-phenylene)-1H-benzimidazole-4,6-disulfonic acid, (1,3,5)-triazine-2,4-bis((4-(2-ethylhexyloxy)-2-hydroxy)phenyl)-6-(4-methoxyphenyl), 2-ethylhexyl 2-cyano-3,3-diphenyl-2-propenoate, glyceryl octanoate, di-p-methoxycinnamic acid, p-aminobenzoic acid and esters thereof, 4-tert-butyl-4′-methoxydibenzoylmethane, 4-(2-β-glucopyranoxy)propoxy-2-hydroxybenzophenone, octyl salicylate, methyl-2,5-diisopropylcinnamic acid, cinoxate, dihydroxydimethoxybenzophenone, disodium salt of 2,2′-dihydroxy-4,4′-dimethoxy-5,5′-disuifobenzophenone, dihydroxybenzophenone, 1,3,4-dimethoxyphenyl-4,4-dimethyl-1,3-pentanedione, 2-ethylhexyl dimethoxybenzylidenedioxoimidazolidinepropionate, methylenebisbenzotriazolyl tetramethylbutylphenol, phenyl dibenzimidazoletetrasulfonate, bis-ethylhexyloxyphenol methoxyphenol triazine, tetrahydroxybenzophenones, terephthalylidenedicamphorsulfonic acid, 2,4,6-tris[4,2-ethylhexyloxycarbonyl)anilino]-1,3,5-triazine, methylbis(trimethylsiloxy)silylisopentyltrimethoxycinnamic acid, amyl p-dimethylaminobenzoate, amyl p-dimethylaminobenzoate, 2-ethylhexyl p-dimethylaminobenzoate, isopropyl-p-methoxycinnamic acid/diisopropylcinnamic acid esters, 2-ethylhexyl-p-methoxycinnamic acid, 2-hydroxy-4-methoxybenzophenone, 2-hydroxy-4-methoxybenzophenone-5-sulfonic acid and the trihydrate, and also 2-hydroxy-4-methoxybenzophenone-5-sulfonate sodium salt and phenylbenzimidazolesulfonic acid.

The amount of the aforementioned sun protection filters (one or more compounds) in the compositions of the present invention is preferably 0.001 to 30.0% by weight, particularly preferably 0.05 to 20.0% by weight and especially preferably 1.0 to 10.0% by weight, based on the total weight of the final composition.

The compositions according to the invention can comprise antioxidants, preferably selected from amino acids (e.g. glycine, histidine, tyrosine, tryptophan) and derivatives thereof, imidazoles (e.g. urocanic acid) and derivatives thereof, peptides such as D,L-carnosine, D-carnosine, L-carnosine and derivatives thereof (e.g. anserine), carotenoids, carotenes (e.g. α-carotene, β-carotene, lycopene) and derivatives thereof, chlorogenic acid and derivatives thereof, lipoic acid and derivatives thereof (e.g. dihydrolipoic acid), aurothioglucose, propylthiouracil and other thiols (e.g. thioredoxin, glutathione, cysteine, cystine, cystamine and the glycosyl, N-acetyl, methyl, ethyl, propyl, amyl, butyl and lauryl, palmitoyl, oleyl, γ-linoleyl, cholesteryl and glyceryl esters thereof), and also salts thereof, dilauryl thiodipropionate, distearyl thiodipropionate, thiodipropionic acid and derivatives thereof (e.g. esters, ethers, peptides, lipids, nucleotides, nucleosides and salts), and also sulfoximine compounds (e.g. buthionine sulfoximines, homocysteine sulfoximine, buthionine sulfones, penta-, hexa-, heptathionine sulfoximine) in very low tolerated doses, also (metal) chelating agents (e.g. α-hydroxy fatty acids, palmitic acid, phytic acid, lactoferrin), α-hydroxy acids (e.g. citric acid, lactic acid, malic acid), humic acid, bile acid, bile extracts, bilirubin, biliverdin, EDTA, EGTA and derivatives thereof, unsaturated fatty acids and derivatives thereof (e.g. γ-linolenic acid, linoleic acid, oleic acid), folic acid and derivatives thereof, ubiquinone and ubiquinol and derivatives thereof, vitamin C and derivatives (e.g. ascorbyl palmitate, Mg ascorbyl phosphate, ascorbyl acetate), tocopherols and derivatives (e.g. vitamin E acetate), vitamin A and derivatives (vitamin A palmitate), and coniferyl benzoate of benzoin resin, rutinic acid and derivatives thereof, α-glycosylrutin, ferulic acid, furfurylideneglucitol, carnosine, butylhydroxytoluene, butylhydroxyanisole, nordihydroguaiacic acid, nordihydroguaiaretic acid, trihydroxybutyrophenone, uric acid and derivatives thereof, mannose and derivatives thereof, zinc and derivatives thereof (e.g. ZnO, ZnSO₄), selenium and derivatives thereof (e.g. selenomethionine), stilbenes and derivatives thereof (e.g. stilbene oxide, trans-stilbene oxide), superoxide dismutase and the derivatives suitable according to the invention (salts, esters, ethers, sugars, nucleotides, nucleosides, peptides and lipids) of these specified substances.

The antioxidants can protect the skin and the hair against oxidative stress. Preferred antioxidants here are vitamin E and derivatives thereof, and vitamin A and derivatives thereof.

The amount of the one or more antioxidants in the compositions according to the invention is preferably 0.001 to 30.0% by weight, particularly preferably 0.05 to 20.0% by weight and especially preferably 1.0 to 10.0% by weight, based on the total weight of the composition.

Furthermore, humectants selected from the sodium salt of 2-pyrrolidone-5-carboxylate (NaPCA), guanidine; glycolic acid and salts thereof, lactic acid and salts thereof, glucosamines and salts thereof, lactamide monoethanolamine, acetamide monoethanolamine, urea, hydroxy acids, panthenol and derivatives thereof, for example D-panthenol (R-2,4-dihydroxy-N-(3-hydroxypropyl)-3,3-dimethylbutamide), D,L-panthenol, calcium pantothenate, panthetine, pantotheine, panthenyl ethyl ether, isopropyl palmitate, glycerol and/or sorbitol can be used, preferably in amounts of from 0.1 to 15.0% by weight and particularly preferably from 0.5 to 5.0% by weight, based on the final compositions.

Additionally, the compositions according to the invention can comprise organic solvents. In principle, suitable organic solvents are all mono- or polyhydric alcohols. Preference is given to using alcohols having 1 to 4 carbon atoms, such as ethanol, propanol, isopropanol, n-butanol, isobutanol, t-butanol, glycerol and mixtures of said alcohols. Further preferred alcohols are polyethylene glycols with a relative molecular mass below 2000. In particular, a use of polyethylene glycol with a relative molecular mass between 200 and 600 and in amounts up to 45% by weight and of polyethylene glycol with a relative molecular mass between 400 and 600 in amounts of from 5.0 to 25.0% by weight is preferred. Further suitable solvents are, for example, triacetin (glycerol triacetate) and 1-methoxy-2-propanol.

The dyes and color pigments present in the compositions according to the invention, both organic and inorganic dyes, are selected from the corresponding positive list of the Cosmetics Regulations or the EU list of cosmetic colorants.

| Chemical or other name | CIN | Color |
|---|---|---|
| Pigment Green | 10006 | green |
| Acid Green 1 | 10020 | green |
| 2,4-Dinitrohydroxynaphthalene-7-sulfonic acid | 10316 | yellow |
| Pigment Yellow 1 | 11680 | yellow |
| Pigment Yellow 3 | 11710 | yellow |
| Pigment Orange 1 | 11725 | orange |
| 2,4-Dihydroxyazobenzene | 11920 | orange |
| Solvent Red 3 | 12010 | red |
| 1-(2′-Chloro-4′-nitro-1′-phenylazo)-2- | 12085 | red |
| hydroxynaphthalene | | |
| Pigment Red 3 | 12120 | red |
| Cerise Red; Sudan Red; Fat Red G | 12150 | red |
| Pigment Red 112 | 12370 | red |
| Pigment Red 7 | 12420 | red |
| Pigment Brown 1 | 12480 | brown |
| 4-(2′-Methoxy-5′-sulfonic acid diethylamide-1′- | 12490 | red |
| phenylazo)-3-hydroxy-5″-chloro-2″,4″-dimethoxy- | | |
| 2-naphthoic acid anilide | | |
| Disperse Yellow 16 | 12700 | yellow |
| 1-(4-Sulfo-1-phenylazo)-4-aminobenzenesulfonic acid | 13015 | yellow |
| 2,4-Dihydroxyazobenzene-4′-sulfonic acid | 14270 | orange |
| 2-(2,4-Dimethylphenylazo-5-sulfonic acid)-1-hydroxy- | 14700 | red |
| naphthalene-4-sulfonic acid | | |
| 2-(4-Sulfo-1-naphthylazo)-1-naphthol-4-sulfonic acid | 14720 | red |
| 2-(6-Sulfo-2,4-xylylazo)-1-naphthol-5-sulfonic acid | 14815 | red |
| 1-(4′-Sulfophenylazo)-2-hydroxynaphthalene | 15510 | orange |
| 1-(2-Sulfonic acid-4-chloro-5-carboxylic acid-1- | 15525 | red |
| phenylazo)-2-hydroxynaphthalene | | |
| 1-(3-Methylphenylazo-4-sulfonic acid)-2- | 15580 | red |
| hydroxynaphthalene | | |
| 1-(4′,(8′)-Sulfonic acid naphthylazo)-2- | 15620 | red |
| hydroxynaphthalene | | |
| 2-Hydroxy-1,2′-azonaphthalene-1′-sulfonic acid | 15630 | red |
| 3-Hydroxy-4-phenylazo-2-naphthylcarboxylic acid | 15800 | red |
| 1-(2-Sulfo-4-methyl-1-phenylazo)-2- | 15850 | red |
| naphthylcarboxylic acid | | |
| 1-(2-Sulfo-4-methyl-5-chloro-1-phenylazo)-2-hydroxy- | 15865 | red |
| naphthalene-3-carboxylic acid | | |
| 1-(2-Sulfo-1-naphthylazo)-2-hydroxynaphthalene- | 15880 | red |
| 3-carboxylic acid | | |
| 1-(3-Sulfo-1-phenylazo)-2-naphthol-6-sulfonic acid | 15980 | orange |
| 1-(4-Sulfo-1-phenylazo)-2-naphthol-6-sulfonic acid | 15985 | yellow |
| Allura Red | 16035 | red |
| 1-(4-Sulfo-1-naphthylazo)-2-naphthol-3,6-disulfonic | 16185 | red |
| acid | | |
| Acid Orange 10 | 16230 | orange |
| 1-(4-Sulfo-1-naphthylazo)-2-naphthol-6,8-disulfonic | 16255 | red |
| acid | | |
| 1-(4-Sulfo-1-naphthylazo)-2-naphthol-3,6,8-trisulfonic | 16290 | red |
| acid | | |
| 8-Amino-2-phenylazo-1-naphthol-3,6-disulfonic acid | 17200 | red |
| Acid Red 1 | 18050 | red |
| Acid Red 155 | 18130 | red |
| Acid Yellow 121 | 18690 | yellow |
| Acid Red 180 | 18736 | red |
| Acid Yellow 11 | 18820 | yellow |
| Acid Yellow 17 | 18965 | yellow |
| 4-(4-Sulfo-1-phenylazo)-1-(4-sulfophenyl)-5-hydroxy- | 19140 | yellow |
| pyrazolone-3-carboxylic acid | | |
| Pigment Yellow 16 | 20040 | yellow |
| 2,6-((4′-Sulfo-2″,4″-dimethyl)bisphenylazo)-1,3- | 20170 | orange |
| dihydroxy-benzene | | |
| Acid Black 1 | 20470 | black |
| Pigment Yellow 13 | 21100 | yellow |
| Pigment Yellow 83 | 21108 | yellow |
| Solvent Yellow | 21230 | yellow |
| Acid Red 163 | 24790 | red |
| Acid Red 73 | 27290 | red |
| 2-[4′-(4″-Sulfo-1″-phenylazo)-7′-sulfo-1′-naphthylazo]- | 27755 | black |
| 1-hydroxy-7-aminonaphthalene-3,6-disulfonic acid | | |
| 4′-[(4″-Sulfo-1″-phenylazo)-7′-sulfo-1′-naphthylazo]- | 28440 | black |
| 1-hydroxy-8-acetylaminonaphthalene-3,5-disulfonic | | |
| acid | | |
| Direct Orange 34, 39, 44, 46, 60 | 40215 | orange |
| Food Yellow | 40800 | orange |
| trans-β-Apo-8′-Carotenealdehyde (C₃₀) | 40820 | orange |
| trans-Apo-8′-Carotenic acid (C₃₀)-ethyl ester | 40825 | orange |
| Canthaxanthin | 40850 | orange |
| Acid Blue 1 | 42045 | blue |
| 2,4-Disulfo-5-hydroxy-4′-4″-bis(diethylamino)- | 42051 | blue |
| triphenyl-carbinol | | |
| 4-[(4-N-Ethyl-p-sulfobenzylamino)phenyl(4-hydroxy- | 42053 | green |
| 2-sulfophenyl)(methylene)-1-(N-ethyl-N-p- | | |
| sulfobenzyl)-2,5-cyclohexadieneimine] | | |
| Acid Blue 7 | 42080 | blue |
| (N-Ethyl-p-sulfobenzylaminophenyl(2-sulfophenyl)- | 42090 | blue |
| methylene(N-ethyl-N-p-sulfobenzyl)cyclohexa- | | |
| dieneimine | | |
| Acid Green 9 | 42100 | green |
| Diethyldisulfobenzyldi-4-amino-2-chlorodi-2-methyl- | 42170 | green |
| fuchsonimmonium | | |
| Basic Violet 14 | 42510 | violet |
| Basic Violet 2 | 42520 | violet |
| 2′-Methyl-4′-(N-ethyl-N-m-sulfobenzyl)amino-4″- | 42735 | blue |
| (N-diethyl)-amino-2-methyl-N-ethyl-N-m- | | |
| sulfobenzylfuchsonimmonium | | |
| 4′-(N-Dimethyl)amino-4″-(N-phenyl)aminonaphtho- | 44045 | blue |
| N-dimethylfuchsonimmonium | | |
| 2-Hydroxy-3,6-disulfo-4,4′-bisdimethylaminonaphtho- | 44090 | green |
| fuchsinimmonium | | |
| Acid red | 45100 | red |
| 3-(2′-Methylphenylamino)-6-(2′-methyl-4′- | 45190 | violet |
| sulfophenylamino)-9-(2″-carboxyphenyl)xanthenium | | |
| salt | | |
| Acid Red 50 | 45220 | red |
| Phenyl-2-oxyfluorone-2-carboxylic acid | 45350 | yellow |
| 4,5-Dibromofluorescein | 45370 | orange |
| 2,4,5,7-Tetrabromofluorescein | 45380 | red |
| Solvent Dye | 45396 | orange |
| Acid Red 98 | 45405 | red |
| 3′,4′,5′,6′-Tetrachloro-2,4,5,7-tetrabromofluorescein | 45410 | red |
| 4,5-Diiodofluorescein | 45425 | red |
| 2,4,5,7-Tetraiodofluorescein | 45430 | red |
| Quinophthalone | 47000 | yellow |
| Quinophthalonedisulfonic acid | 47005 | yellow |
| Acid Violet 50 | 50325 | violet |
| Acid Black 2 | 50420 | black |
| Pigment Violet 23 | 51319 | violet |
| 1,2-Dioxyanthraquinone, calcium-aluminum complex | 58000 | red |
| 3-Oxypyrene-5,8,10-sulfonic acid | 59040 | green |
| 1-Hydroxy-4-N-phenylaminoanthraquinone | 60724 | violet |
| 1-Hydroxy-4-(4′-methylphenylamino)anthraquinone | 60725 | violet |
| Acid Violet 23 | 60730 | violet |
| 1,4-Di(4′-methylphenylamino)anthraquinone | 61565 | green |
| 1,4-Bis(o-sulfo-p-toluidine)anthraquinone | 61570 | green |
| Acid Blue 80 | 61585 | blue |
| Acid Blue 62 | 62045 | blue |
| N,N′-Dihydro-1,2,1′,2′-anthraquinoneazine | 69800 | blue |
| Vat Blue 6; Pigment Blue 64 | 69825 | blue |
| Vat Orange 7 | 71105 | orange |
| Indigo | 73000 | blue |
| Indigodisulfonic acid | 73015 | blue |
| 4,4′-Dimethyl-6,6′-dichlorothioindigo | 73360 | red |
| 5,5′-Dichloro-7,7′-dimethylthioindigo | 73385 | violet |
| Quinacridone Violet 19 | 73900 | violet |
| Pigment Red 122 | 73915 | red |
| Pigment Blue 16 | 74100 | blue |
| Phthalocyanine | 74160 | blue |
| Direct Blue 86 | 74180 | blue |
| Chlorinated phthalocyanines | 74260 | green |
| Natural Yellow 6,19; Natural Red 1 | 75100 | yellow |
| Bixin, Nor-Bixin | 75120 | orange |
| Lycopene | 75125 | yellow |
| trans-alpha, beta- or gamma-Carotene | 75130 | orange |
| Keto- and/or hydroxyl derivatives of carotene | 75135 | yellow |
| Guanine or pearlescent agents | 75170 | white |
| 1,7-Bis(4-hydroxy-3-methoxyphenyl)-1,6-heptadiene- | 75300 | yellow |
| 3,5-dione | | |
| Complex salt (Na,Al,Ca) of carminic acid | 75470 | red |
| Chlorophyll a and b; copper compounds of the | 75810 | green |
| chlorophylls and chlorophyllines | | |
| Aluminum | 77000 | white |
| Aluminum hydrate | 77002 | white |
| Water-containing aluminum silicates | 77004 | white |
| Ultramarine | 77007 | blue |
| Pigment Red 101 and 102 | 77015 | red |
| Barium sulfate | 77120 | white |
| Bismuth oxychloride and its mixtures with mica | 77163 | white |
| Calcium carbonate | 77220 | white |
| Calcium sulfate | 77231 | white |
| Carbon | 77266 | black |
| Pigment Black 9 | 77267 | black |
| Carbo medicinalis vegetabilis | 77268:1 | black |
| Chromic oxide | 77288 | green |
| Chromic oxide, water-containing | 77289 | green |
| Pigment Blue 28, Pigment Green 14 | 77346 | green |
| Pigment Metal 2 | 77400 | brown |
| Gold | 77480 | brown |
| Iron oxides and hydroxides | 77489 | orange |
| Iron oxides and hydroxides | 77491 | red |
| Hydrated iron oxide | 77492 | yellow |
| Iron oxide | 77499 | black |
| Mixtures of iron(II) and iron(III) hexacyanoferrate | 77510 | blue |
| Pigment White 18 | 77713 | white |
| Manganese ammonium diphosphate | 77742 | violet |
| Manganese phosphate; Mn₃(PO₄)₂•7H₂O | 77745 | red |
| Silver | 77820 | white |
| Titanium dioxide and its mixtures with mica | 77891 | white |
| Zinc oxide | 77947 | white |
| 6,7-Dimethyl-9-(1′-D-ribityl)isoalloxazine, lactoflavin | | yellow |
| Caramel | | brown |
| Capsanthin, Capsorubin | | orange |
| Betanine | | red |
| Benzopyrilium salts, anthocyanines | | red |
| Aluminum, zinc, magnesium and calcium stearate | | white |
| Bromothymol Blue | | blue |
| Bromocresol Green | | green |
| Acid Red 195 | | red |

Oil-soluble natural dyes, such as, for example, paprika extracts, β-carotene and cochineal are furthermore advantageous.

Also advantageously used are pearlescent pigments, e.g. pearl essence (guanine/hypoxanthine mixed crystals from fish scales) and mother of pearl (ground mussel shells), monocrystalline pearlescent pigments such as, for example, bismuth oxychloride (BiOCl), layer substrate pigments, e.g. mica/metal oxide, silver-white pearlescent pigments from TiO₂, interference pigments (TiO₂, variable layer thickness), color luster pigments (Fe₂O₃) and combination pigments (TiO₂/Fe₂O₃, TiO₂/Cr₂O₃, TiO₂/Prussian blue, TiO₂/carmine).

Effect pigments within the context of the present invention are understood as meaning pigments which due to their refraction properties produce special optical effects. Effect pigments impart to the treated surface (skin, hair, mucous membrane) luster or glitter effects or can visually conceal unevenness of the skin and skin wrinkles by means of diffuse light scattering. As a particular embodiment of the effect pigments, interference pigments are preferred. Particularly suitable effect pigments are, for example, mica particles which are coated with at least one metal oxide. Besides mica, a sheet silicate, silica gel and other SiO₂ modifications are also suitable as carriers. A metal oxide frequently used for coating is, for example, titanium oxide, to which, if desired, iron oxide can be admixed. By means of the size and shape (e.g. spherical, ellipsoidal, flat, even, uneven) of the pigment particles and by means of the thickness of the oxide coating, the reflection properties can be influenced. Other metal oxides, e.g. bismuth oxychloride (BiOCl), and the oxides of, for example, titanium, in particular the TiO₂ modifications anatase and rutile, aluminum, tantalum, niobium, zirconium and hafnium can also be used. Effect pigments can also be prepared using magnesium fluoride (MgF₂) and calcium fluoride (fluorspar, CaF₂).

The effects can be controlled both by means of the particle size and by means of the particle size distribution of the pigment ensemble. Suitable particle size distributions extend, for example, from 2-50 μm, 5-25 μm, 5-40 μm, 5-60 μm, 5-95 μm, 5-100 μm, 10-60 μm, 10-100 μm, 10-125 μm, 20-100 μm, 20-150 μm, and <15 μm. A wider particle size distribution, for example of 20-150 μm, produces glittering effects, whereas a narrower particle size distribution of <15 μm provides for a uniform silky appearance.

The compositions of the present invention comprise effect pigments preferably in amounts from 0.1% to 20.0% by weight, more preferably from 0.5% to 10.0% by weight and even more preferably from 1.0% to 5.0% by weight, all based on the total weight of the composition.

Preference as deodorizing substances is given to allantoin and bisabolol. These are preferably used in amounts from 0.0001% to 10.0% by weight.

Fragrance and/or perfume oils which may be used are individual odorant compounds, e.g. the synthetic products of the ester, ether, aldehyde, ketone, alcohol and hydrocarbon types. Odorant compounds of the ester type are, for example, benzyl acetate, phenoxyethyl isobutyrate, p-tert-butylcyclohexyl acetate, linalyl acetate, dimethylbenzylcarbinyl acetate, phenylethyl acetate, linalyl benzoate, benzyl formate, ethylmethylphenyl glycinate, allyl cyclohexyl propionate, styrallyl propionate and benzyl salicylate. The ethers include, for example, benzyl ethyl ethers, the aldehydes include, for example, the linear alkanals havina 8 to 18 carbon atoms, citral, citronellal, citronellyloxyacetaldehyde, cyclamenaldehyde, hydroxycitronellal, lilial and bourgeonal, the ketones include, for example, the ionones, alpha-isomethylionone and methyl cedryl ketone, the alcohols include anethol, citronellol, eugenol, geraniol, linalool, phenylethyl alcohol and terpineol, and the hydrocarbons include primarily the terpenes and balsams. Preference is given to using mixtures of different odorants which together produce a pleasing scent note.

Perfume oils can also comprise natural odorant mixtures, as are accessible from vegetable or animal sources, e.g. pine oil, citrus oil, jasmine oil, lily oil, rose oil or ylang-ylang oil. Essential oils of relatively low volatility, which in most cases are used as aromatic components, are also suitable as perfume oils, e.g. sage oil, chamomile oil, clove oil, melissa oil, mint oil, cinnamon leaf oil, linden blossom oil, juniperberry oil, vetiver oil, olibanum oil, galbanum oil and ladanum oil.

Preferably suitable as pearlizing component are fatty acid monoalkanolamides, fatty acid dialkanolamides, monoesters or diesters of alkylene glycols, in particular ethylene glycol and/or propylene glycol or oligomers thereof, with higher fatty acids, such as, for example, palmitic acid, stearic acid and behenic acid, monoesters or polyesters of glycerol with carboxylic acids, fatty acids and metal salts thereof, ketosulfones or mixtures of the specified compounds. Particular preference is given to ethylene glycol distearates and/or polyethylene glycol distearates having on average 3 glycol units.

When the compositions according to the invention comprise pearlizing compounds, these are preferably present in the compositions according to the invention in an amount of from 0.1 to 15.0% by weight and particularly preferably in an amount of from 1.0 to 10.0% by weight.

The acids or alkalis used for adjusting the pH are preferably mineral acids, in particular HCl, inorganic bases, in particular NaOH or KOH, and organic acids, in particular citric acid.

The following examples and applications are intended to further elucidate the invention without, however, limiting it thereto. All percentages are percent (%) by weight.

### PREPARATION EXAMPLES

### General Operating Procedure

In the preparation of the phosphoric esters of the present invention, phosphoric acid (85% strength), polyol and fatty alcohol alkoxylate are used in a certain molar ratio. For this purpose, all starting materials are initially charged in a stirred apparatus equipped with heating mantle, separator with condenser and vacuum connection. The mixture is heated to 100° C. and three times evacuated down to 100 mbar and subsequently refilled with nitrogen. After a further 4 hours of inertization (nitrogen being introduced at 20 liters/hour) at 100° C., the batch is heated to 230° C. while nitrogen is being introduced and esterified (water removed in the separator). The reaction times are 24 to 42 hours (reckoned from 230° C. esterification temperature), particularly 40 hours. The residual acid number is then <15 mg KOH/g. After the reaction has ended, the product is cooled to 80° C. and poured into a dish and the solidified melt is comminuted.

### Example 1

The general preparation procedure was followed to prepare an ester from 18.2 g of phosphoric acid, 179.6 g of pentaerythritol+100 mol of ethylene oxide and 482.6 g of Ceteareth-25 (C_{16/18} fatty alcohol+25 mol of ethylene oxide, Genapol® T 250) in a molar ratio of 4:1:8. The residual acid number was 5.7 mg KOH/g, corresponding to 86% conversion. The ester is a white wax having a melting point of about 40° C.

### Example 2

The general operating procedure was followed to prepare an ester from 22.8 g of phosphoric acid, 224.5 g of pentaerythritol+100 mol of ethylene oxide and 294.8 g of Ceteareth-11 (C_{16/18} fatty alcohol+11 mol of ethylene oxide, Genapol® T 110) in a molar ratio of 4:1:8. The residual acid number was 12.1 mg KOH/g, corresponding to 80% conversion. The ester is a white wax having a melting point of about 38° C.

Viscosity Measurements in Aqueous Solutions

The viscosities of the phosphoric esters of Examples 1 and 2 compared with the commercial products Crothix (PEG-150 Pentaerythrityl Tetrastearate), Genapol® DAT 100 (PEG-150 Polyglyceryl-2 Tristearate) and Rewopal® PEG 6000 DS (PEG-150 Distearate) were measured (in each case 6% by weight of product in water).

The viscosity was measured at 20° C. with an RVT type Brookfield viscometer at 20 rpm immediately after preparation of the aqueous solutions (“Viscosity as-prepared”) and after 3 months' storage at room temperature (RT).

TABLE 1
Viscosities in aqueous solutions

| | Viscosity | | |
|---|---|---|---|
| | as- | | Viscosity after 3 months' |
| | prepared | | storage at RT |
| Product | [mPa · s] | Clarity | [mPa · s] |
| Example 1 | 164000 | clear | 88000 |
| Example 2 | 98000 | clear | 60500 |
| PEG 150 pentaerythrityl | 6650 | cloudy | 990 |
| tetrastearate | | | |
| PEG 150 polyglyceryl | 65 | cloudy | n.d. |
| 2-tristearate | | | |
| PEG 150 distearate | 3900 | cloudy | n.d. |

PEG: polyethylene glycol;
RT: room temperature;
n.d. not determined

The results from Table 1 show that the inventive phosphoric esters according to Examples 1 and 2 provide clear gels of high viscosity in water compared with the comparative examples. In addition the viscosity reduction in storage is distinctly less than in the case of PEG 150 pentaerythrityl tetrastearate for example.

Viscosity Measurements in Aqueous-Surfactant Solutions

The viscosities of the phosphoric esters of Examples 1 and 2 compared with the commercial products Crothix (PEG-150 Pentaerythrityl Tetrastearate), Genapol® DAT 100 (PEG-150 Polyglyceryl-2 Tristearate) and Rewopal® PEG 6000 DS (PEG-150 Distearate) were measured (in each case 1% by weight of product in an aqueous solution of sodium laureth ether sulfate with 2 ethylene oxide units (INCI: Sodium Laureth Sulfate):

Cocamidopropylbetaine at a ratio of 8:2, with an active substance content of Sodium Laureth Sulfate/Cocamidopropylbetaine of 15% by weight in water; pH 4-4.4. The viscosity was measured at 20° C. with an RVT type Brookfield viscometer at 20 rpm immediately after preparation of the aqueous-surfactant solutions (“Viscosity as-prepared”) and after 4 months' storage at 50° C.

TABLE 2
Viscosities in aqueous-surfactant solutions

| | Viscosity as- | Viscosity after 4 months' |
|---|---|---|
| | prepared | storage at 50° C. |
| Product | [mPa · s] | [mPa · s] |
| Example 1 | 406000 | 45000 |
| Example 2 | 69000 | 73000 |
| PEG 150 pentaerythrityl | 20000 | 80 |
| tetrastearate | | |
| PEG 150 polyglyceryl | 23100 | 110 |
| 2-tristearate | | |
| PEG 150 distearate | 2750 | 45 |

PEG: polyethylene glycol

The results from Table 2 show that the inventive phosphoric esters according to Examples 1 and 2 give gels of high viscosity in surfactant (compared with the comparative examples) which, unlike the comparative examples, do not lose viscosity in the course of storage.

### FORMULATION EXAMPLES

### Formulation Example 1

### Facial Cleansing Foam

| | | |
|---|---|---|
| A | stearic acid | 1.60% |
| | myristic acid | 1.80% |
| | lauric acid | 0.70% |
| | Tegin M | 0.50% |
| | Glyceryl Stearate | |
| | palmitic acid | 0.70% |
| B | water | ad 100.00% |
| C | potassium hydroxide | 0.70% |
| | phosphoric ester of Example 1 | 1.10% |

Preparation:
I Melt A at 80° C.
II Dissolve C in B with stirring and at 60° C., then add to I.
III Cool down with stirring.

### Formulation Example 2

### Cream Rinse

| | | | |
|---|---|---|---|
| A | Genamin ® CTAC | (Clariant) | 6.00% |
| | Cetrimonium Chloride | | |
| | Hostacerin ® DGL | (Clariant) | 1.50% |
| | PEG-10 Diglyceryl-2 Laurate | | |
| | Cetylstearyl Alcohol | | 1.70% |
| | paraffin oil | | 1.00% |
| B | water | | ad 100.00% |
| C | phosphoric ester of Example 2 | | 0.80% |
| D | perfume | | 0.30% |
| | panthenol | | 0.30% |
| | preservative | | q.s. |
| | dye | | q.s. |

Preparation:
I Dissolve A at 75° C.
II Dissolve C in B. with stirring at 60° C.
III Add II to I with stirring. Stir until cold.
IV At 40° C. add the components of D.
V Adjust the pH to 4.

### Formulation Example 3

### Light Leave On for Hair Tips

| | | | |
|---|---|---|---|
| A | SilCare ® Silicone 41M15 | (Clariant) | 0.30% |
| | Caprylyl Methicone | | |
| B | Genapol ® LA 070 | (Clariant) | 8.00% |
| | Laureth-7 | | |
| C | water | | ad 100% |
| D | phosphoric ester of Example 2 | | 1.50% |
| E | Biobranil | | 0.50% |
| | Soybean (Glycine Soja) Oil and Wheat | | |
| | (Triticum Vulgare) Bran Lipids | | |
| | glycerol | | 2.00% |
| | panthenol | | 0.50% |
| F | SilCare ® Silicone SEA | (Clariant) | 0.50% |
| | Trideceth-9 PG Amodimethicone and | | |
| | Trideceth-12 | | |
| | Genamin ® CTAC | (Clariant) | 2.00% |
| | Cetrimonium Chloride | | |
| | Nipaguard ® DMDMH | (Clariant) | 0.20% |
| | DMDMH Hydantoin | | |

Preparation:
I Solubilize A in B.
II Dissolve Din C with stirring at 60° C.
III Add E to II and stir until the solution is clear, then add to I.
IV Add F to III.

### Formulation Example 4

### Hydrogen Peroxide Gel

| | | | |
|---|---|---|---|
| A | phosphoric ester of Example 3 | | 3.00% |
| | Genapol ® T 250 | (Clariant) | 2.00% |
| | Ceteareth-25 | | |
| B | water | | ad 100.00% |
| C | phosphoric acid | | 0.04% |
| | sodium dihydrogen phosphate | | 1.00% |
| D | hydrogen peroxide 30% strength | | 18.00% |

Preparation:
I Dissolve A in B with stirring and heating to 50° C.
II Add C at 25° C.
III Add D at room temperature.

### Formulation Example 5

### Deodorant Gel

| | | | |
|---|---|---|---|
| A | Octopirox ® | (Clariant) | 0.10% |
| | Piroctone Olamine | | |
| B | Emulsogen ® HCP 049 | (Clariant) | 10.00% |
| | PEG-40 Hydrogenated Castor Oil and | | |
| | Propylene Glycol | | |
| | perfume | | 0.20% |
| C | water | | ad 100.00% |
| D | phosphoric ester of Example 2 | | 2.00% |
| E | citric acid | | q.s. |

Preparation:
I Dissolve A in B.
II Dissolve D in C with stirring and slight heating, then add II to I.
III If necessary, adjust the pH to 6.0 with E.

### Formulation Example 6

### Make-Up Remover

| | | | |
|---|---|---|---|
| A | Velsan ® P8-3 | (Clariant) | 5.00% |
| | Isopropyl C12-15 Pareth-9 Carboxylate | | |
| B | Hostapon ® CGN | (Clariant) | 2.00% |
| | Sodium Cocoyl Glutamate | | |
| | Genagen ® CAB | (Clariant) | 3.00% |
| | Cocamidopropyl Betaine | | |
| | Allantoin | (Clariant) | 0.30% |
| | Aristoflex ® PEA | (Clariant) | 1.00% |
| | Polypropylene Terephthalate | | |
| | 1.6 Hexanediol | | 2.00% |
| | 1.2 Propanediol | | 2.00% |
| | Polyglycol 400 | (Clariant) | 2.00% |
| | PEG-8 | | |
| | panthenol | | 0.50% |
| | Lutrol F 127 | | 3.00% |
| | Poloxamer 407 | | |
| | preservative | | q.s. |
| C | phosphoric ester of Example 1 | | 0.60% |
| D | water | | ad 100.00% |
| E | Genapol ® LA 070 | (Clariant) | 2.00% |
| | Laureth-7 | | |

Preparation:
I A little at a time add the components of B to A and stir until a clear solution forms.
II Dissolve C in D with stirring and slight heating, add II to I.
III Stir E into I.

### Formulation Example 7

### Whitening Gel

| A | water | | ad 100.00% |
|---|---|---|---|
| | arginine | | 1.10% |
| | phosphoric ester of Example 1 | | 3.50% |
| B | dipropylene glycol | | 8.00% |
| | Genapol ® C 100 | (Clariant) | 0.3.5% |
| | Coceth-10 | | |
| | Sodium citrate*2H₂O | | 0.09% |
| | citric acid 10.0% | | 0.10% |
| | Nipagin ® M | (Clariant) | 0.20% |
| | Methylparaben ascorbic acid 2-glucoside | | 2.00% |

Preparation:
I Mix the components of A and dissolve with stirring and slight heating.
II Add the components of B to I and dissolve. If necessary, heat the formulation slightly.

### Formulation Example 8

### Facial Toner

| | | | |
|---|---|---|---|
| A | glycerol | | 8.00% |
| | Polyglycol 400 | (Clariant) | 5.00% |
| | PEG-8 | | |
| | panthenol | | 0.50% |
| | perfume | | 0.20% |
| | alcohol | | 8.00% |
| | preservative | | q.s. |
| | Allantoin | (Clariant) | 0.10% |
| | Niacinamide | | 0.10% |
| | Extrapon Hamamelis | | 1.00% |
| | water, Witch Hazel Distillate, SD Alcohol | | |
| | 39-C, Butylene Glycol | | |
| B | water | | ad 100% |
| C | phosphoric ester of Example 1 | | 2.50% |

Preparation:
I Dissolve C in B with stirring and slight heating.
II Add the components of A to I and stir until formulation is homogeneous.

### Formulation Example 9

### Hair Shampoo

| | | | |
|---|---|---|---|
| A | Genapol ® LRO liquid | (Clariant) | 30.00% |
| | Sodium Laureth Sulfate | | |
| | Hostapon ® CGN | (Clariant) | 5.00% |
| | Sodium Cocoyl Glutamate | | |
| | perfume | | 0.30% |
| B | water | | ad 100.00% |
| C | phosphoric ester of Example 2 | | 1.55% |
| | preservative | | q.s. |
| | dye | | q.s. |
| | Genagen ® CAB | (Clariant) | 8.00% |
| | Cocamidopropyl Betaine | | |

Preparation:
I Dissolve C in B with stirring and heating to 50° C.
II A little at a time stir the components of A into I.
III If necessary, adjust the pH.

### Formulation Example 10

### Foam Bath

| | | | |
|---|---|---|---|
| A | Genapol ® LRO liquid | (Clariant) | 60.00% |
| | Sodium Laureth Sulfate | | |
| B | Medialan ® LD | (Clariant) | 8.00% |
| | Sodium Lauroyl Sarcosinate | | |
| | perfume | | 1.50% |
| | Velsan ® CG 070 | (Clariant) | 5.00% |
| | PEG-7 Glyceryl Cocoate | | |
| C | phosphoric ester of Example 2 | | 1.20% |
| D | water | | ad 100% |
| E | dye | | q.s. |
| | preservative | | q.s. |
| | Genagen ® CAB | (Clariant) | 6.00% |
| | Cocamidopropyl Betaine | | |

Preparation:
I A little at a time stir the components of B into A.
II Dissolve C in D with stirring and heating to 50° C.
III Add I to II.
VI Stir E into III.
V If necessary, adjust the pH.

### Formulation Example 11

### O/W Skin Milk

| | | | |
|---|---|---|---|
| A | Hostacerin ® DGI | (Clariant) | 2.00% |
| | Polyglyceryl-2 Sesquiisostearate | | |
| | Isopropyl palmitate | | 4.00% |
| | Octyldodecanol | | 4.00% |
| | Nipaguard ® PDU | (Clariant) | q.s. |
| | Propylene Glycol (and) Diazolidinyl Urea | | |
| | (and) Methylparaben (and) Propylparaben | | |
| B | Aristoflex ® AVC | (Clariant) | 1.20% |
| | Ammonium Acryloyldimethyltaurate/VP | | |
| | Copolymer | | |
| C | Hostapon ® CGN | (Clariant) | 0.60% |
| | Sodium Cocoyl Glutamate | | |
| | water | | ad 100% |
| D | phosphoric ester of Example 2 | | 1.30% |
| E | perfume | | 0.40% |

Preparation:
I Dissolve D in C with stirring and heating to 50° C.
II Add B to A, then add I and stir thoroughly.
III Add E to II.
IV Finally homogenize the formulation.

### Formulation Example 12

### Antiperspirant Roll-On

| | | | |
|---|---|---|---|
| A | phosphoric ester of Example 2 | | 1.50% |
| B | water | | ad 100.00% |
| C | Locron ® L | (Clariant) | 20.00% |
| | Aluminum Chlorohydrate | | |
| D | Genapol ® T 250 | (Clariant) | 5.00% |
| | Ceteareth-25 | | |
| | Butylene Glycol | | 3.00% |
| | Cetiol OE | | 1.00% |
| | Dicaprylyl Ether | | |
| | Glyceryl Isostearate | | 2.00% |
| E | SilCare ® Silicone SEA | (Clariant) | 0.50% |
| | Trideceth-9 PG Amodimethicone and | | |
| | Trideceth-12 | | |

Preparation:
I Dissolve A in B with stirring and heating to 60° C.
II Add C to I.
III Melt D at 50° C. and add II and stir until a clear solution has formed.
IV Add E at 30° C.

Formulation Example 12 displayed a distinct reduction of white residues on the clothing after use of the roll-on on the skin compared with the same formulation but without phosphoric ester according to Example 2.

### Formulation Example 13

### Vitamin C Gel

| | | | |
|---|---|---|---|
| A | phosphoric ester of Example 2 | | 1.30% |
| | Genapol ® T 250 | (Clariant) | 2.00% |
| | Ceteareth-25 | | |
| B | water | | ad 100.00% |
| C | ascorbic acid | | 3.00% |
| D | Aristoflex AVC | (Clariant) | 0.80% |
| | Ammonium Acryloyldimethyltaurate/VP | | |
| | Copolymer | | |

Preparation:
I Dissolve A in B with stirring at 50° C.
II Stir C into I at room temperature.
III Add D and stir until a homogeneous gel has formed.

### Formulation Example 14

### Shower Bath

| | | | |
|---|---|---|---|
| A | phosphoric ester of Example 2 | (Clariant) | 2.50% |
| | Aristoflex ® PEA | (Clariant) | 2.00% |
| | Polypropylene-Terephthalate | | |
| B | water | | ad 100% |
| C | Genapol ® LRO liquid | (Clariant) | 30.00% |
| | Sodium Laureth Sulfate | | |
| | Genapol ® LA 030 | (Clariant) | 1.50% |
| | Laureth-3 | | |
| | Hostapon ® CLG | (Clariant) | 5.00% |
| | Sodium Lauroyl Glutamate | | |
| | Genagen ® KB | (Clariant) | 6.00% |
| | Coco Betaine | | |
| | perfume | | 0.30% |
| | dye | | q.s. |
| | preservative | | q.s. |

Preparation:
I Dissolve A in B at 50° C.
II Stir the components of C into I in succession.
III Adjust pH if necessary.

### Formulation Example 15

### Facial Anti-Aging Cream Gel

| | | | |
|---|---|---|---|
| A | phosphoric ester of Example 1 | (Clariant) | 1.00% |
| B | water | | ad 100.00% |
| C | paraffin oil | | 5.00% |
| | SilCare ® Silicone 31M50 | (Clariant) | 3.00% |
| | Caprylyl Trimethicone | | |
| D | Aristoflex ® AVC | (Clariant) | 1.80% |
| | Ammonium Acryloyldimethyltaurate/VP | | |
| | Copolymer | | |
| E | glycolic acid 30%* | | 6.00% |
| | Phenonip ® | (Clariant) | 0.50% |
| | Phenoxyethanol (and) Methylparaben | | |
| | (and) Ethylparaben (and) Butylparaben | | |
| | (and) Propylparaben (and) | | |
| | Isobutylparaben | | |
| F | Genapol ® LA 070 | (Clariant) | 2.00% |
| | Laureth-7 | | |

*neutralized to pH 4 with NaOH.

Preparation:
I Dissolve A in B with stirring at 50° C.
II Stir D into C.
III Stir I into II and stir until a homogeneous gel has formed.
IV Add E into III.
V Stir F into IV and stir until cream gel is homogeneous.

### Formulation Example 16

### O/W Self-Tanning Cream

| | | | |
|---|---|---|---|
| A | Hostaphat ® CC 100 | (Clariant) | 1.0% |
| | Cetyl Phosphate | | |
| | Glyceryl Stearate | | 0.5% |
| | Cetearyl Alcohol | | 0.5% |
| | paraffin oil | | 8.0% |
| | isopropyl palmitate | | 7.0% |
| | SilCare ® Silicone 41M15 | (Clariant) | 1.0% |
| | Caprylyl Methicone | | |
| B | Aristoflex ® AVC | (Clariant) | 1.2% |
| | Ammonium Acryloyldimethyltaurate/VP | | |
| | Copolymer | | |
| C | water | | ad 100% |
| D | phosphoric ester of Example 2 | (Clariant) | 1.0% |
| E | Hostapon ® CLG | (Clariant) | 0.5% |
| | Sodium Lauroyl Glutamate | | |
| | glycerol | | 5.0% |
| F | Tocopheryl Acetate | | 1.0% |
| | Fragrance | | 0.2% |
| | Preservative | | q.s. |
| G | Dihydroxyacetone | | 5.0% |
| H | water | | 8.0% |
| I | sodium hydroxide (10% in water) | | q.s. |

Preparation:
I Melt A at 80° C.
II Stir B into A.
III Dissolve D in C at 50° C., then add E.
IV Stir III into II.
V Add F at room temperature.
VI Dissolve G in H and stir into V.
VII Adjust pH to 4-5 with I if necessary.

### Formulation Example 17

### O/W Sunscreen Milk

| | | | |
|---|---|---|---|
| A | Hostaphat ® CK 100 | (Clariant) | 2.00% |
| | Potassium Cetyl Phosphate | | |
| | SilCare ® Silicone 41M15 | (Clariant) | 1.00% |
| | Caprylyl Methicone | | |
| | stearic acid | | 0.50% |
| | Cetyl Alcohol | | 0.50% |
| | Cutina ® GMS | | 1.00% |
| | Glyceryl Stearate | | |
| | Cetiol ® SN | | 4.00% |
| | Cetearyl Isononanoate | | |
| | Velsan ® CCT | (Clariant) | 4.00% |
| | Caprylic/Capric Triglyceride | | |
| | Neo ® Heliopan BB | | 1.50% |
| | Benzophenone - 3 | | |
| | Eusolex ® 6300 | | 4.00% |
| | 4-Methylbenzylidene Camphor | | |
| B | Aristoflex ® AVC | (Clariant) | 0.40% |
| | Ammonium Acryloyldimethyltaurate/VP | | |
| | Copolymer | | |
| C | water | | ad 100% |
| D | phosphoric ester of Example 2 | (Clariant) | 0.80% |
| E | glycerol | | 3.00% |
| | Eusolex ® 232 | | 2.00% |
| | Phenylbenzimidazole Sulfonic Acid | | |
| | Tris(hydroxymethyl)aminomethane | | 1.10% |
| | Tromethamine | | |
| F | Tocopheryl Acetate | | 0.50% |
| | Phenonip ® | (Clariant) | 0.50% |
| | Phenoxyethanol (and) Methylparaben | | |
| | (and) Butylparaben (and) Ethylparaben | | |
| | (and) Propylparaben | | |
| | Fragrance | | 0.40% |

Preparation:
I Melt A at 80° C., then add B.
II Dissolve D in C at 60° C.
III Stir E into II.
IV Dissolve III in I.
V Add F into IV at 35° C.

### Formulation Example 18

### Facial Anti-Aging Gel

| | | | |
|---|---|---|---|
| A | Genapol ® T 250 | (Clariant) | 1.00% |
| | Ceteareth-25 | | |
| | phosphoric ester of Example 2 | (Clariant) | 1.40% |
| B | water | | ad 100% |
| C | Aristoflex ® AVC | (Clariant) | 2.00% |
| | Ammonium Acryloyldimethyltaurate/VP | | |
| | Copolymer | | |
| D | Glycolic acid 30%* | | 6.00% |
| | Phenonip ® | (Clariant) | 0.50% |
| | Phenoxyethanol (and) Methylparaben | | |
| | (and) Ethylparaben (and) Butylparaben (and) | | |
| | Propylparaben (and) Isobutylparaben | | |

*neutralized to pH 4 with NaOH.

Preparation:
I Dissolve A in B with stirring at 50° C.
II Add C and stir until a homogeneous gel has formed.
III Add D and stir until the gel is again homogeneous.

1. A phosphoric ester comprising
A) at least one structural unit derived from substances of component a), the substances of component a) being selected from orthophosphoric acid and at least one of its derivatives
B) at least one structural unit derived from substances of component b), the substances of component b) being selected from at least one compound of formula (I)
R²—O—(CH₂CH₂O)ᵤ(C₃H₆O)ᵥ(DO)_{w}—H (I)
wherein
R² is a linear or branched, saturated alkyl group having 6 to 30 carbon atoms, or is a linear or branched mono- or polyunsaturated alkenyl group having 6 to 30 carbon atoms,
D is a linear or branched saturated alkylene group having 4 to 20 carbon atoms, is a linear or branched mono- or polyunsaturated alkenylene group having 4 to 20 carbon atoms or is —CH(phenyl)CH₂—,
u is a number from 0 to 200,
v is a number from 0 to 100,
w is a number from 0 to 100, and
where the groups CH₂CH₂O, C₃H₆O and DO from the compounds of formula (I) can be arranged blocklike or randomly distributed, and
C) at least one structural unit derived from substances of component c), the substances of component c) being selected from at least one polyol having more than 2 OH groups which may also have at least one alkoxylate group and where the alkoxylate group are each constructed of one or more units selected from the group consisting of CH₂CH₂O—, C₃H₆O— and C₄H₈— units which may each be arranged blocklike or randomly distributed within the alkoxylate groups,
and where the phosphoric esters contain at least 2 phosphorus atoms per molecule which are bridged via a structural unit derived from the at least one polyol having more than 2 OH groups or derived from the at least one polyol having more than 2 OH groups having at least one of the alkoxylate group.

2. A phosphoric ester according to claim 1 wherein the substances of component c) are selected from the group consisting of glycerol, diglycerol, polyglycerol, pentaerythritol, dipentaerythritol, pentaerythritol oligomers, trimethylolpropane, threitol, erythritol, adonitol, arabitol, xylitol, mannitol, sorbitol, inositol, glucose, mannose, fructose, sorbose, arabinose, xylose, ribose, mannopyranose, galactopyranose, glucopyranose, maltose, sucrose, amino sugar, ascorbic acid, glucamides and gluconamides, which may further have at least one alkoxylate group and where the alkoxylate group is each constructed of at least one unit selected from the group consisting of CH₂CH₂O—, C₃H₆O— and C₄H₈O— units which each may be arranged blocklike or randomly distributed within the alkoxylate groups.

3. A phosphoric ester according to claim 1, wherein the substances of component c) are selected from the group consisting of pentaerythritol, glycerol and diglycerol, which may further have at least one alkoxylate group and where the alkoxylate groups are each constructed of at least one unit selected from the group consisting of CH₂CH₂O—, C₃H₆O— and C₄H₈O— units which each may be arranged blocklike or randomly distributed within the alkoxylate groups.

4. A phosphoric ester according to claim 1, wherein the substances of component c) have at least one alkoxylate group.

5. A phosphoric ester according to claim 4 wherein the at least one alkoxylate group of the substances of component c) consist of CH₂CH₂O-groups and the number of CH₂CH₂O— groups per polyol molecule having more than 2 OH groups is in the range from 1 to 150.

6. A phosphoric ester according to claim 1, wherein the substances of component b) are selected from at least one compound of formula (II),
R²—O—(CH₂CH₂O)ᵤ₁(C₃H₆O)ᵥ₁—H (II)
where
R² is a linear or branched saturated alkyl group having 6 to 30 carbon atoms, or is a linear or branched mono- or polyunsaturated alkenyl group having 6 to 30 carbon atoms,
u1 is a number from 1 to 200, and
v1 is a number from 1 to 100,
and wherein the CH₂CH₂O— and C₃H₆O— units may be arranged blocklike or randomly distributed.

7. A phosphoric ester according to claim 1, wherein the substances of component b) are selected from at least one compound of formula (III),
R²—O—(CH₂CH₂O)ᵤ₁—H (III)
where
R² is a linear or branched saturated alkyl group having 6 to 30 carbon atoms, or is a linear or branched mono- or polyunsaturated alkenyl group having 6 to 30 carbon atoms, and
u1 is a number from 1 to 200.

8. A phosphoric ester according to claim 1, wherein the at least one structural unit derived from the at least one compound of formula (I) is such a structural unit wherein u is a number from 1 to 200, v and w are 0 and the radical R²—O— is derived from at least one alcohol selected from the group consisting of octanol, decanol, dodecanol, tetradecanol, hexadecanol, octadecanol, eicosanol, behenyl alcohol, fatty alcohols having C-chain cuts between 8 and 22, branched fatty alcohols, and monounsaturated fatty alcohols.

9. A phosphoric ester according to claim 1, wherein the at least one structural unit derived from the at least one compound of formula (I) are structural units derived from C_{16/18} fatty alcohol ethoxylates having 10-50 ethylene oxide units.

10. A phosphoric ester according to claim 1, wherein the total number in the phosphoric ester of ethylene oxide units in the structural units derived from the substances of component b) and the substances of component c) is together in the range from 30 to 100 derived from the compounds of formula (I).

11. A phosphoric ester according to claim 1, wherein it is produced from the reaction of
a) 5 to 10 mol of a C₁₂-C₂₂ fatty alcohol ethoxylate,
b) 1 mol of a polyol selected from the group consisting of pentaerythritol, glycerol and diglycerol wherein each polyol is ethoxylated with 50 to 150 ethylene oxide units, and
c) 2 to 5 mol of orthophosphoric acid or at least one of its derivatives.

12. A phosphoric ester according to claim 1, wherein it is produced from the reaction of
a) 6 to 10 mol, of a C₁₂-C₂₂ fatty alcohol ethoxylate, having 10-50 ethylene oxide units,
b) 1 mol of pentaerythritol ethoxylated with 50 to 150 ethylene oxide units, and
c) 3 to 5 mol, of orthophosphoric acid or at least one of its derivatives.

13. A phosphoric ester according to claim 1, wherein at least 75% of the maximum number of the esterifiable functions theoretically obtainable from the substances of component a) in the phosphoric ester are in an esterified state.

14. A process for preparing a phosphoric ester according to claim 1, comprising the step of reacting a phosphoric acid component selected from orthophosphoric acid and one of its derivatives with an alcohol component and a polyol having more than 2 OH groups or a corresponding polyol comprising alkoxylate groups at temperatures of 150 to 250° C.

15. A process according to claim 14 wherein the phosphoric acid component is a substance selected from the group consisting of orthophosphoric acid, polyphosphoric acid and tetraphosphorus decaoxide.

16. A cosmetic, pharmaceutical or dermatological composition, comprising at least one phosphoric ester according to claim 1.

17. A composition according to claim 16, wherein the composition is in the form of an aqueous, aqueous-alcoholic or aqueous-surfactant composition, in the form of an emulsion, in the form of a suspension, in the form of a dispersion, in the form of a powder or in the form of a spray.

18. A composition according to claim 16 having a pH in the range from 2 to 10.

19. A composition according to claim 16 further comprising at least one electrolyte.

20. A composition according to claim 19 wherein the content of the at least one electrolyte is from 0.1% to 20.0% by weight, based on the entire composition.

21. A composition according to claim 16, further comprising hydrogen peroxide or hydrogen peroxide releasers.

22. A composition according to claim 16, further comprising at least one surfactant.

23. A composition comprising the at least one phosphoric ester according to claim 1 in an amount of 0.01% to 10.0% by weight, based on the final composition.

24. A thickener, consistency regulator, emulsifier, sensory additive, solubilizer, dispersant, glidant, adhesive or stabilizer comprising at least one phosphoric ester according to claim 1.

25. A deodorant or antiperspirant formulation comprising at least one phosphoric ester according to claim 1.

26. A deodorant or antiperspirant formulation comprising aluminum chlorohydrate or aluminum-zirconium complex salts, for reducing the formation of white residues on the clothing after using the deodorant or antiperspirant formulations on the skin and at least one phosphoric ester according to claim 1.
